(19) United States
(12) Patent Application Publication
(10) Pub. No.: US 2003/0078399 A1
(43) Pub. Date: Apr. 24, 2003

(54) Nucleic acid sequence encoding ovarian antigen, CA125, and uses thereof

(75) Inventors: Kenneth O. Lloyd, Bronx, NY (US); Beatrice W.T. Yin, Forest Hills, NY (US)

Correspondence Address: Albert Wai-Kit Chan, Law Offices of Albert Wai-Kit Chan, LLC, World Plaza, Suite 604, 141-07 20th Avenue, Whitestone, NY, 11357, US

(73) Assignee: Sloan-Kettering Institute for Cancer Research

(21) Appl. No.: 10/142,515

(22) Filed: May 9, 2002

Related U.S. Application Data
(60) Provisional application No. 60/290,480, filed on May 11, 2001.

(51) Int. Cl. C07H 21/02; C07H 21/04

(52) U.S. Cl. 536/23.1

(57) ABSTRACT

The present invention provides an isolated nucleic acid molecule comprising sequences encoding the CA125 protein or a portion thereof. This invention also provides a method to detect ovarian cancer in a subject. Furthermore, this invention provides a method for the diagnosis of a cancer which expresses CA125 by detecting CA125-expressing cells in the blood or other fluids of patients. This invention also provides a method of producing CA125 protein. Finally, this invention provides a method to treat or prevent cancer using a vaccine comprising CA125 nucleic acid or protein.

This application claims benefit of U.S. Patent Application No. 60/290,480, Filed on May 11, 2001, the content of which is incorporated here into this application.

The invention disclosed herein was made with government support under NIH Grants No. CA52477 and CA08748, from the United States Department of Health and Human Services. Accordingly, the U.S. Government has certain rights in this invention.

Throughout this application, various references are referred to. Disclosures of these publications in their entireties are hereby incorporated by reference into this application to more fully describe the state of the art to which this invention pertains.

### BACKGROUND OF THE INVENTION

CA125 antigen is a serum marker that is used routinely in gynecologic practice to monitor patients with ovarian cancer. It is a mullerian duct differentiation antigen that is overexpressed in epithelial ovarian cancer cells and secreted into the blood, although its expression is not entirely confined to ovarian cancer. CA125 was first identified by Bast and Knapp (1) in 1981 by a monoclonal antibody (OC125) that had been developed from mice immunized with an ovarian cancer cell line. These investigators subsequently developed a radio-immunoassay for the antigen and showed that serum CA125 levels are elevated in about 80% of patients with epithelial ovarian cancer (EOC)¹ but in less than 1% of healthy women (2). Numerous studies since that time have confirmed the usefulness of CA125 levels in monitoring the progress of patients with EOC (3-6). Most reports indicate that a rise in CA125 levels precedes clinical detection by about 3 months. During chemotherapy, changes in serum CA125 levels correlate with the course of the disease. CA125 is being used in the inventors' Medical Center, and elsewhere, as a surrogate marker for clinical response in phase II trials of new drugs. On the other hand, CA125 is not useful in the initial diagnosis of EOC because of its elevation in a number of benign conditions (3, 7). Despite this limitation, CA125 is considered to be one of the best available cancer serum markers, however more information on its molecular nature is needed to fully explore its potential.

Although CA125 antigen was first detected over 20 years ago, very little is known about its biochemistry and genetics. Most biochemical studies have concluded that CA125 is a high molecular weight glycoprotein, although estimates of its size range from 200 to 2000 kDa with smaller “subunits” being described by some investigators (8-13). Most studies have shown that CA125 is a mucin-type molecule, but others have claimed that it is a typical glycoprotein with asparagine-linked sugar chains (14). Another study claimed that CA125 is a glycosyl-phosphoinositol-linked glycoprotein (11). Thus, no consensus emerged from these studies concerning the biochemical nature of this antigen. Recently, however, our studies have strongly indicated that CA125 is a typical mucin molecule with a high carbohydrate content and a preponderance of serine and threonine-linked (O-linked) glycan chains (15, 16). Possibly because of the mucinous nature of CA125 its peptide moiety has been very difficult to clone. The only published study on this topic (17) described the isolation of a novel cDNA, later termed NBR-1 (18), but this species does not seem to have any of the biochemical characteristics expected for CA125 and may, in fact, be a transcription factor. Using a rabbit antiserum to purified CA125 we have now cloned, by expression cloning, a long partial cDNA sequence corresponding to a new mucin species (designated CA125/MUC16) that is a strong candidate for being the peptide core of the CA125 antigen.

### SUMMARY OF THE INVENTION

The invention disclosed herein provides an isolated nucleic acid molecule comprising sequences encoding the CA125 protein or a portion thereof. This invention also provides the gene encoding the CA125 protein.

In addition, this invention provides a vaccine for cancer which expresses CA125 protein comprising an appropriate amount of the isolated nucleic acid molecules which, when expressed, are capable of producing a product which induces an immune response to CA125 protein. This invention also provides a vaccine for cancer which expresses CA125 protein comprising an appropriate amount of a substance which induces an immune response to CA125 protein. This invention also provides a method for the diagnosis of a cancer which expresses CA125 by detecting CA125-expressing cells in the blood or other fluids of patients based on the nucleic acid sequence which encodes CA125. Furthermore, this invention provides a method for monitoring the therapy of a cancer which expresses CA125 by measuring the expression of CA125-expressing cells in the blood or other fluids of patients based on the nucleic acid sequence which encodes CA125, a decrease of either the number of CA125-expressing cells or level of protein expression in the cell, indicating the success of the therapy.

In addition, this invention provides a method of producing CA125 protein comprising steps of: a) constructing a vector adapted for expression in a cell which comprises the regulatory elements necessary for expression of nucleic acid in the cell operatively linked to the nucleic acid encoding the CA125 protein so as to permit expression thereof; b) placing the cells of step (a) under conditions allowing the expression of the CA125 protein; and c) recovering the CA125 protein so expressed.

Finally, this invention provides a nonhuman organism, wherein the expression of CA125 is inhibited.

### DETAILED DESCRIPTION OF THE FIGURES

### First Series of Experiments

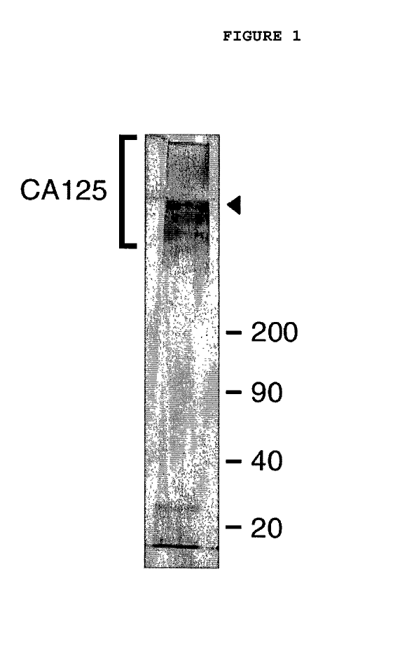

FIG. 1. SDS-PAGE analysis of purified CA125 sample. The gel (3% stacking gel and 5% separating gel) was run under reducing conditions and stained with silver reagent. The arrowhead indicates the interface between the stacking and separating gels. The migration positions of molecular weight markers (in kDa) are shown on the right hand side. The bracket indicates the region of the gel used to immunize a rabbit to produce the polyclonal anti-CA125 serum.

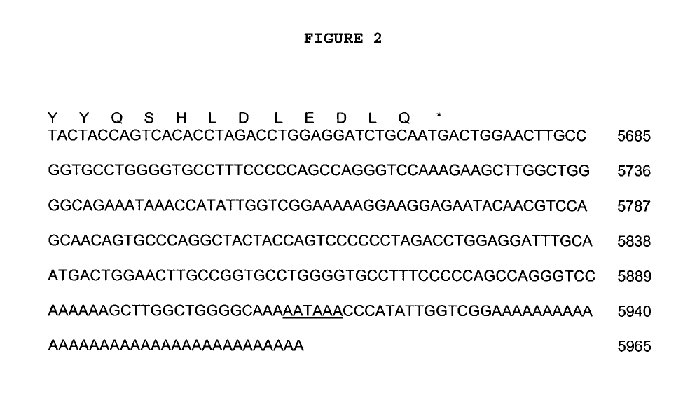

FIG. 2. Nucleotide sequence at 3′ end of the B4 clone of CA125/MUC16. The nucleotide and amino acid sequence for B4 (CA125/MUC16) have been deposited in the GenBank™ under accession number AF361486. Abbreviations: EOC: epithelial ovarian cancer; mAb: monoclonal antibody; TR: tandem repeat; PBS: phosphate buffered saline. * indicates a stop codon. A polyadenylation signal sequence is underlined.

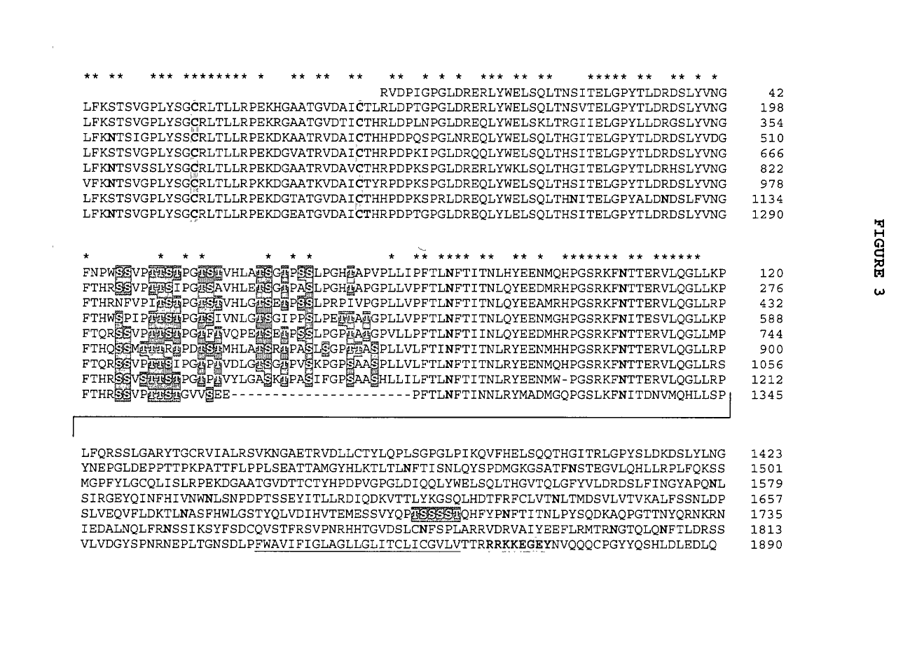

FIG. 3. Deduced amino acid sequence of CA125/MUC16 (B4) organized to indicate the regions of homology in the tandem repeats. Clustered serine and threonine residues are highlighted in white/shade and conserved cysteine residues in bold/shade. Potential N-linked glycosylation sites (Asn) are indicated in bold type. The possible transmembrane region is underlined and the consensus tyrosine phosphorylation motif is indicated in regular/shade. * indicates residues that are perfectly conserved, except in the last repeat sequence. - indicates gaps introduced to preserve the best homology in the repeats.

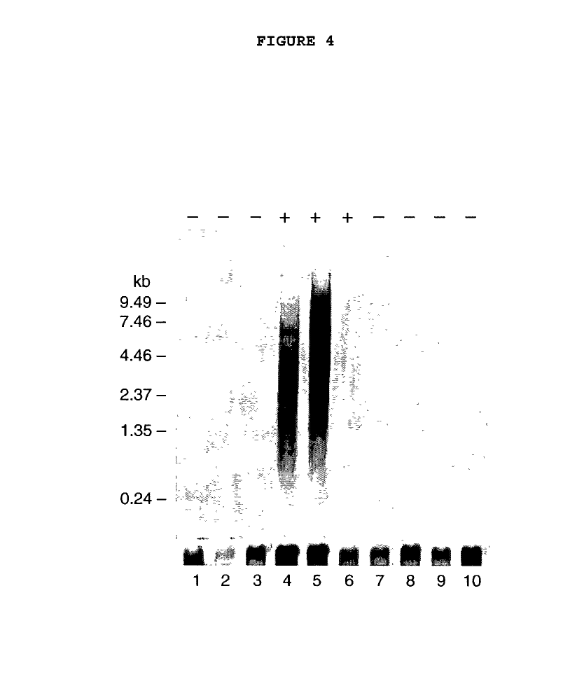

FIG. 4. Northern blot analysis of expression of CA125/MUC16 in cancer cell lines. The blot was probed with a biotin-labeled probe (B53) from the tandem repeat region. 1: SW626 (ovarian cancer); 2: 2774 (ovarian cancer); 3: SK-OV-3 (ovarian cancer); 4: SK-OV-8 (ovarian cancer); 5: OVCAR-3 (ovarian cancer); 6: COLO316 (ovarian cancer); 7: MCF-7 (breast cancer); 8: IMR-3 (neuroblastoma); 9: MKN45 (gastric cancer); 10: MCA (sarcoma). Indicated on the top of the figure (+ or − ) is the expression of CA125 in the cell line as determined by reactivity with anti-CA125 antibodies. The end-point titers for these cell lines with mAb OC125 were 1- <1:500; 2- <1:500; 3- <1:500; 4- 1 : 128,000; 5- >1 : 256,000; 6- 1:4000; 7- <1:500; 8- <1:500; 9- <1:500; 10- <1:500. Screening with mAb VK-8 gave similar results. The result of probing the blot with a β-actin probe is shown in the lower half of the figure. Size standards are indicated on the left side of the gel.

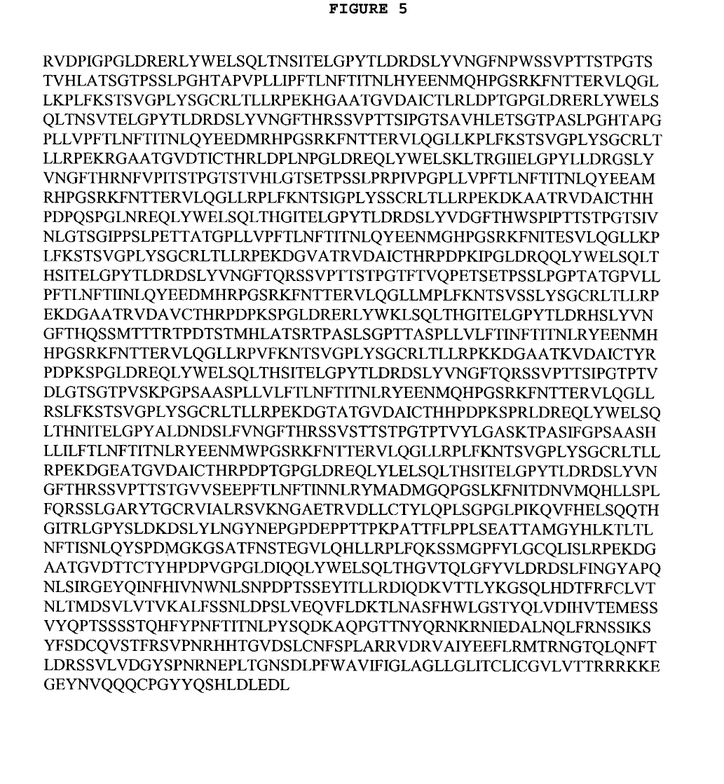

FIG. 5. Deduced amino acid sequence of B4 polynucleotide (CA125).

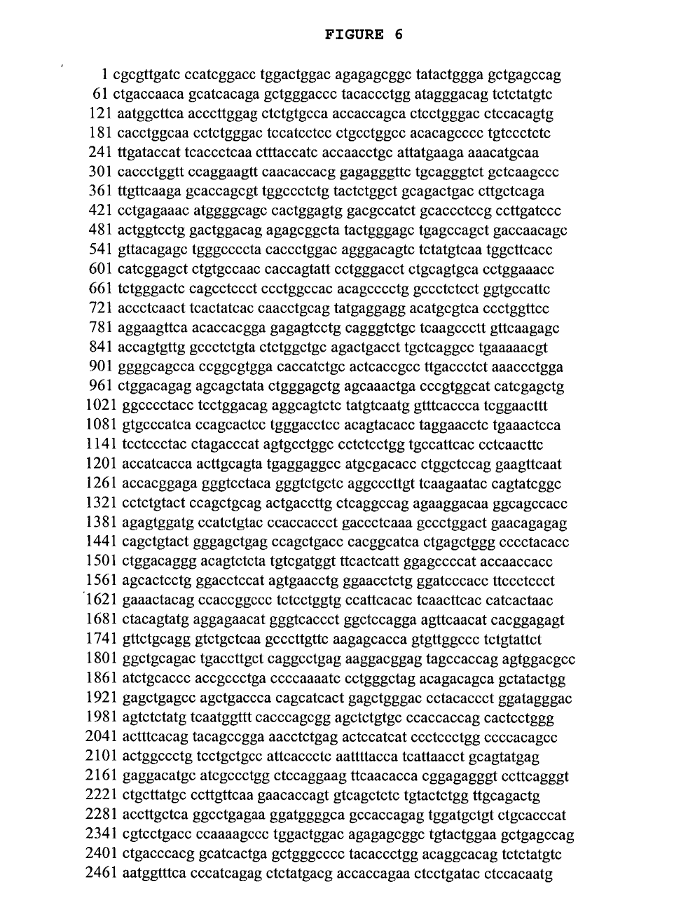

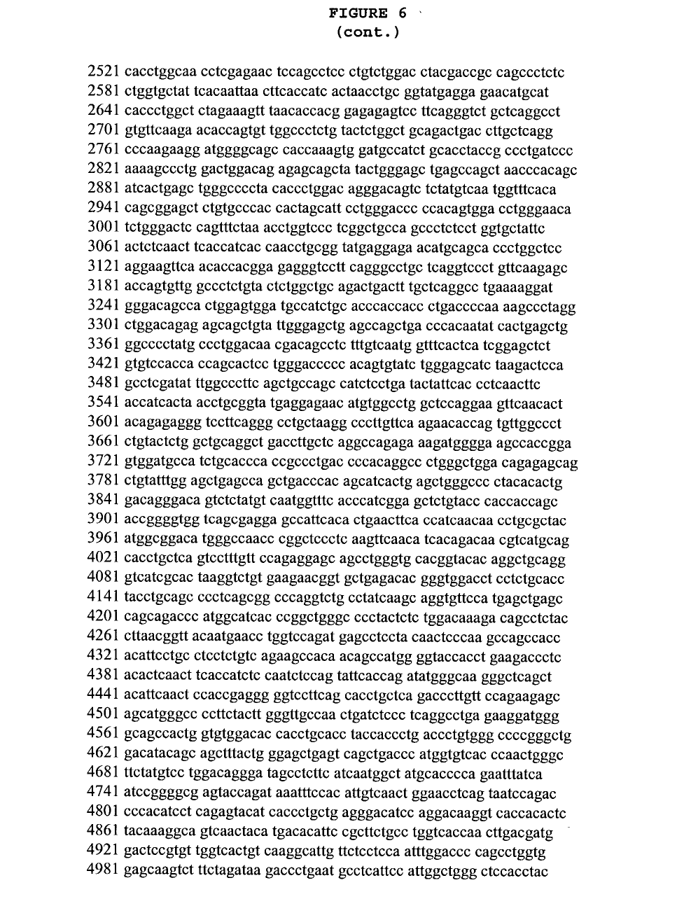

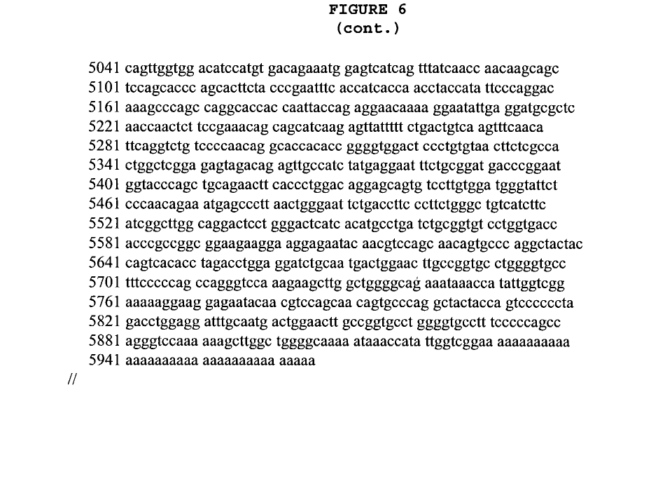

FIG. 6. Nucleotide sequence of B4 polynucleotide (CA125).

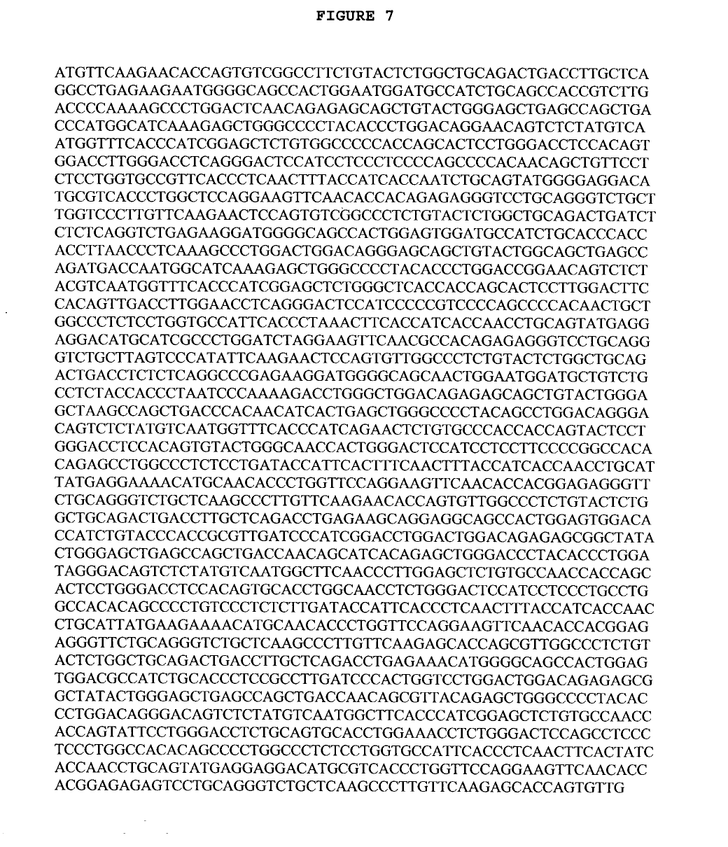

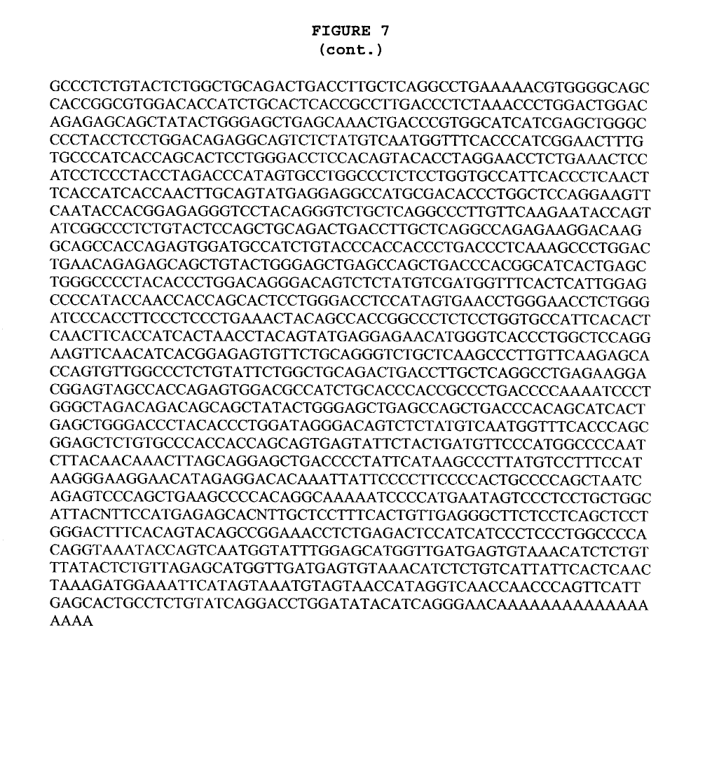

FIG. 7. Nucleotide sequence of B30 polynucleotide coding for a different portion of the CA125 gene.

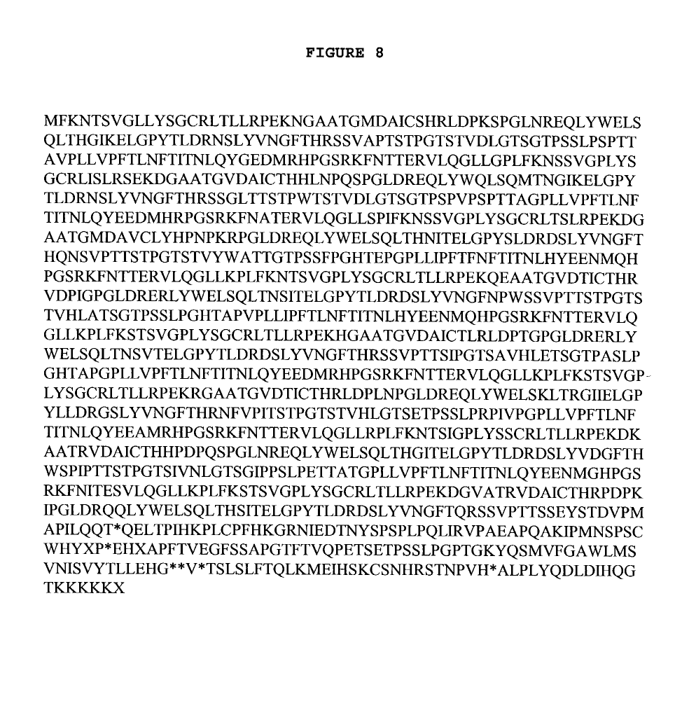

FIG. 8. Deduced amino acid sequence of B30 polynucleotide corresponding to a different portion of the CA125 gene.

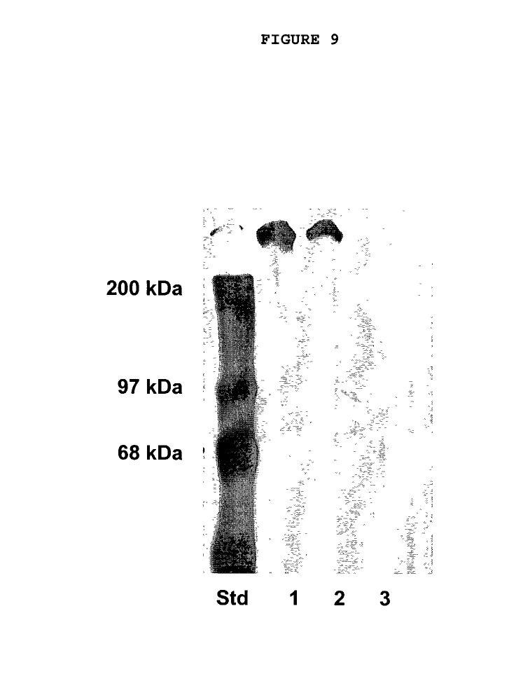

FIG. 9. Expression analysis of CA125 nucleotide clone. This figure is the result of an expression experiment that confirms that the sequence actually codes for CA125, as recognized by standard antibodies.

### Second Series of Experiments

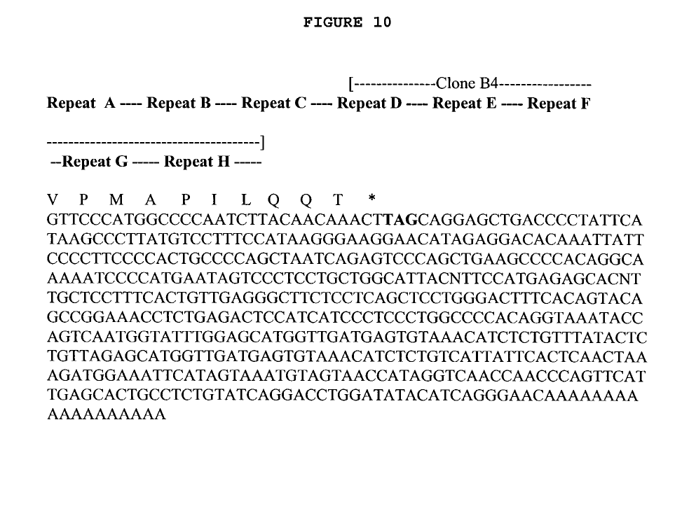

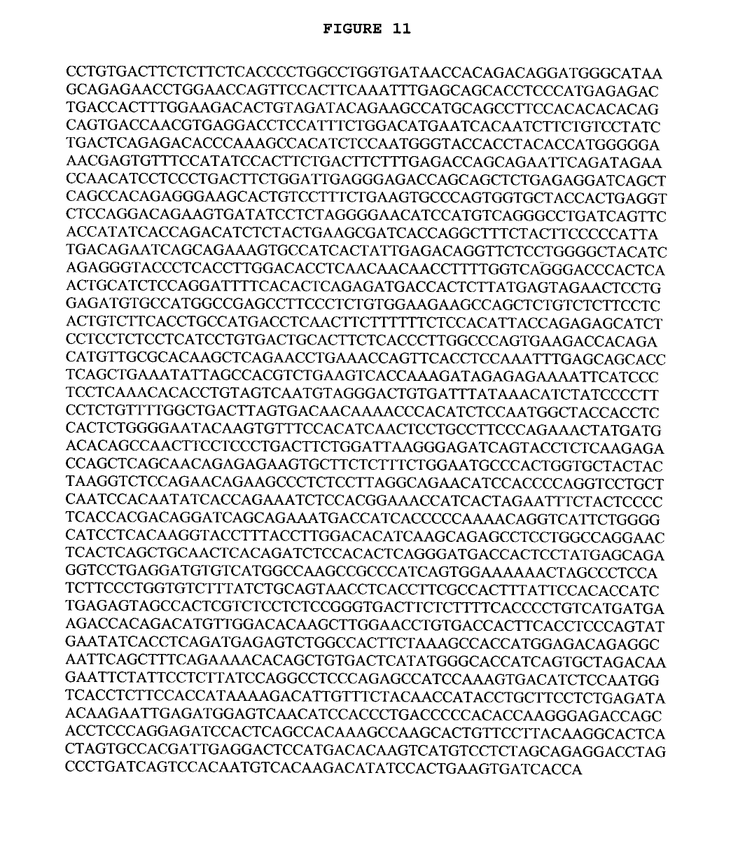

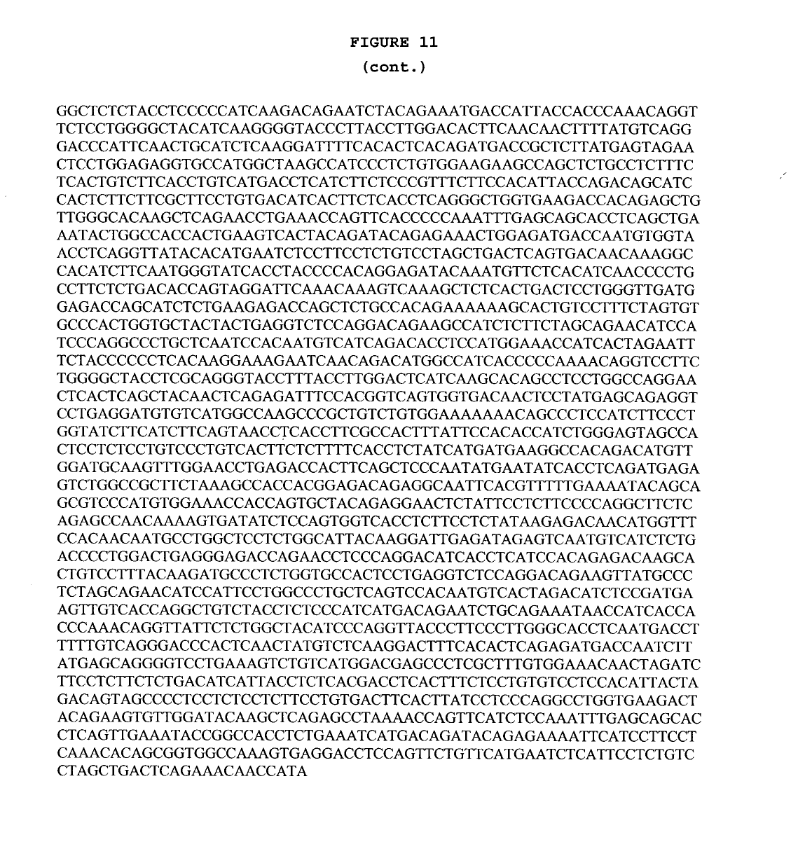

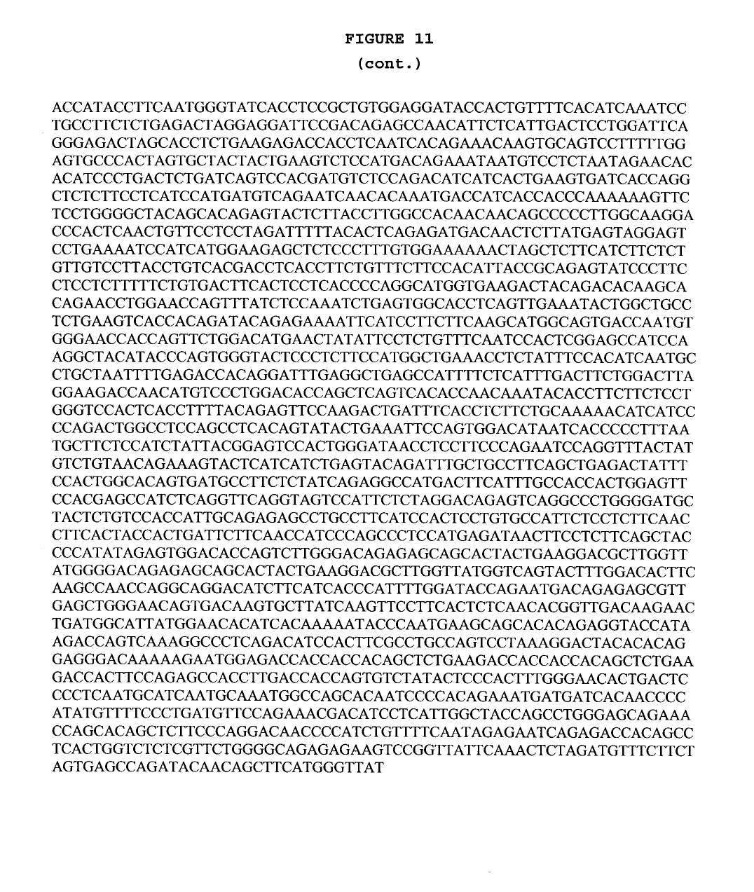

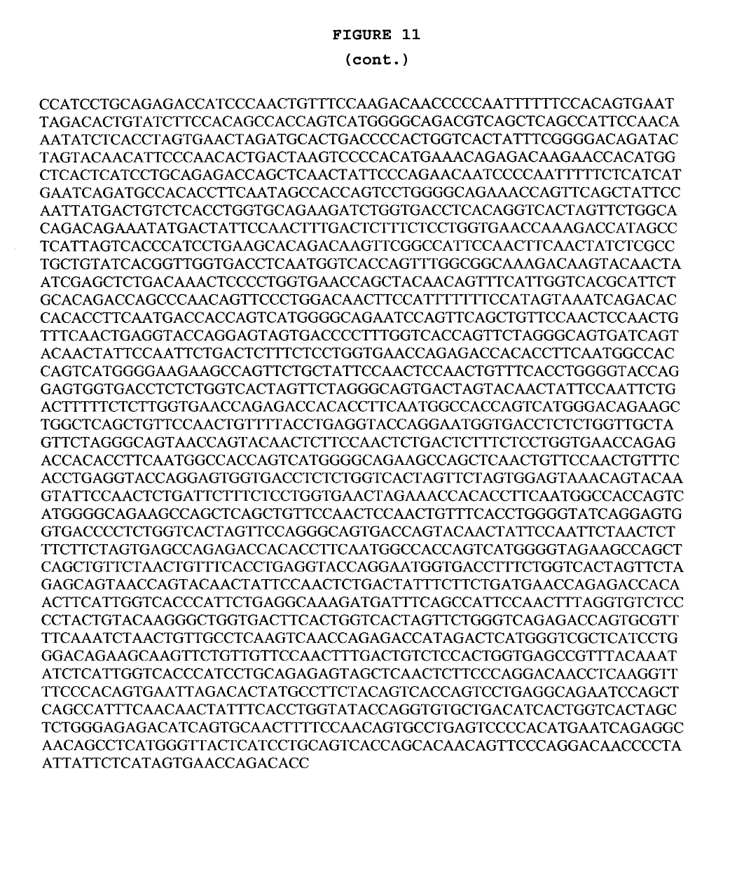

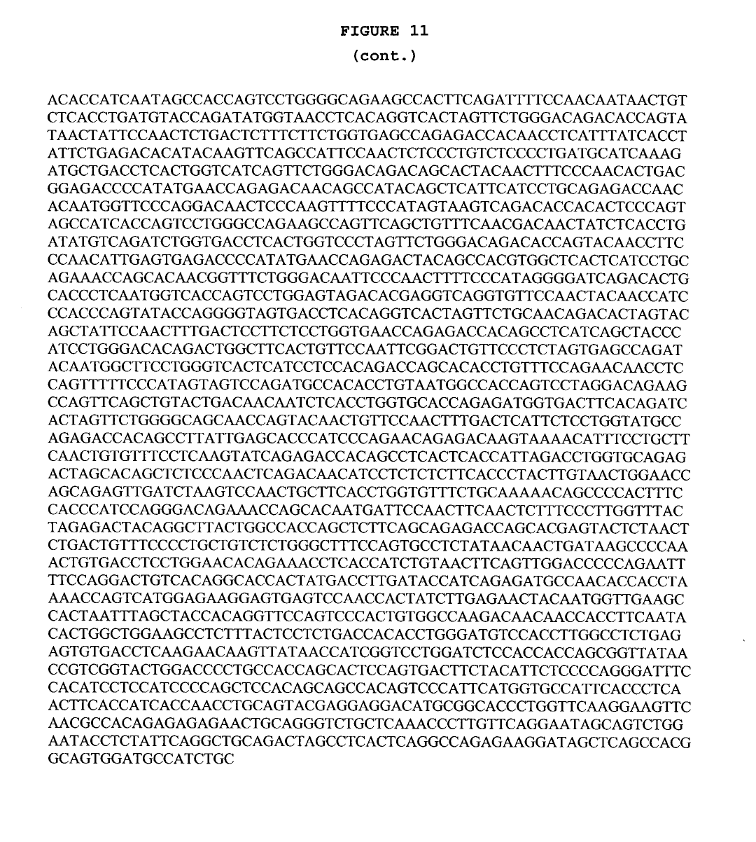

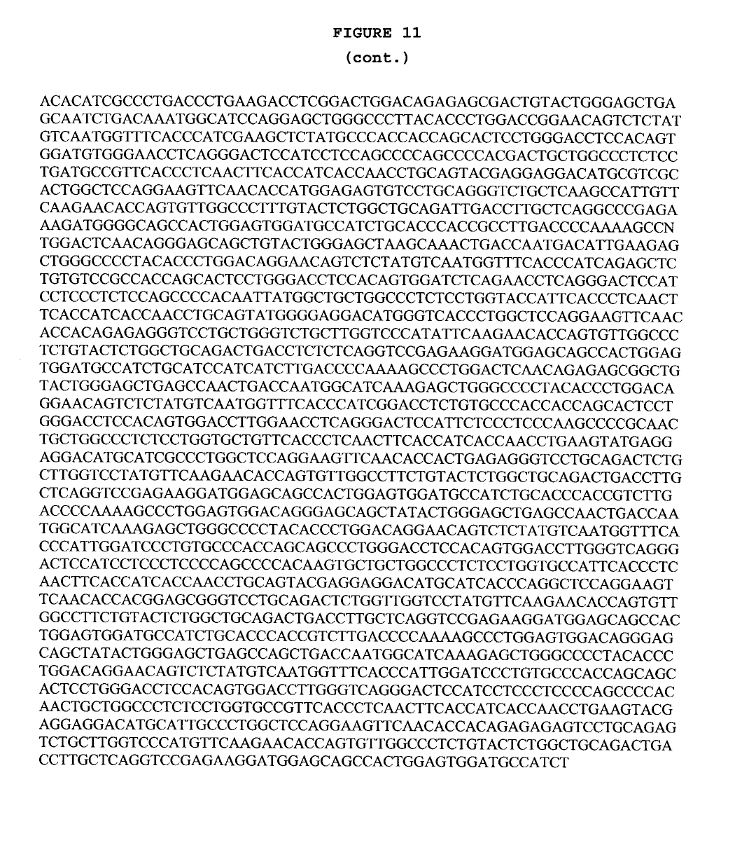

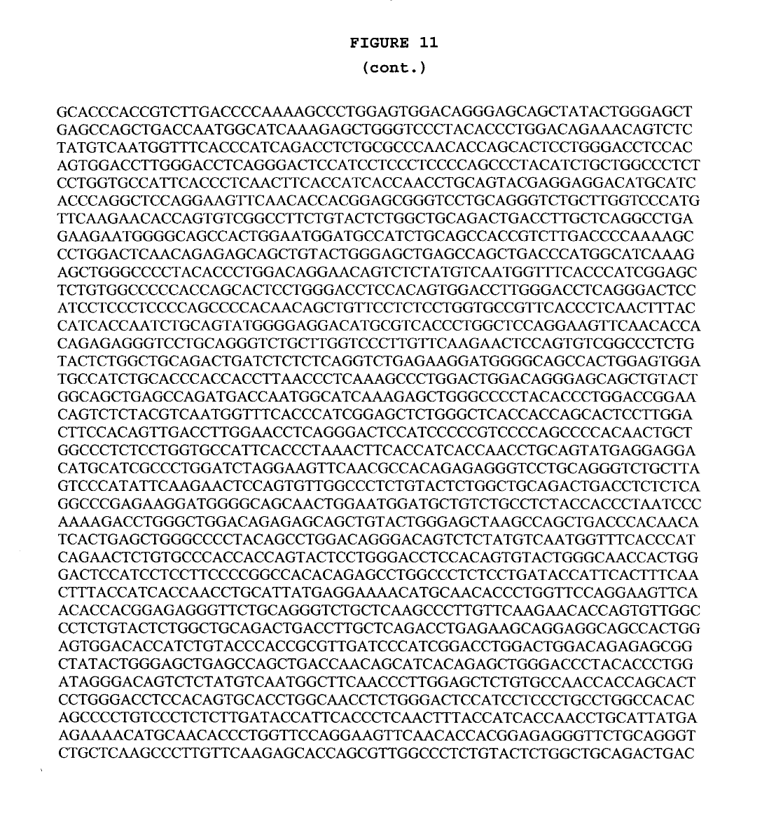

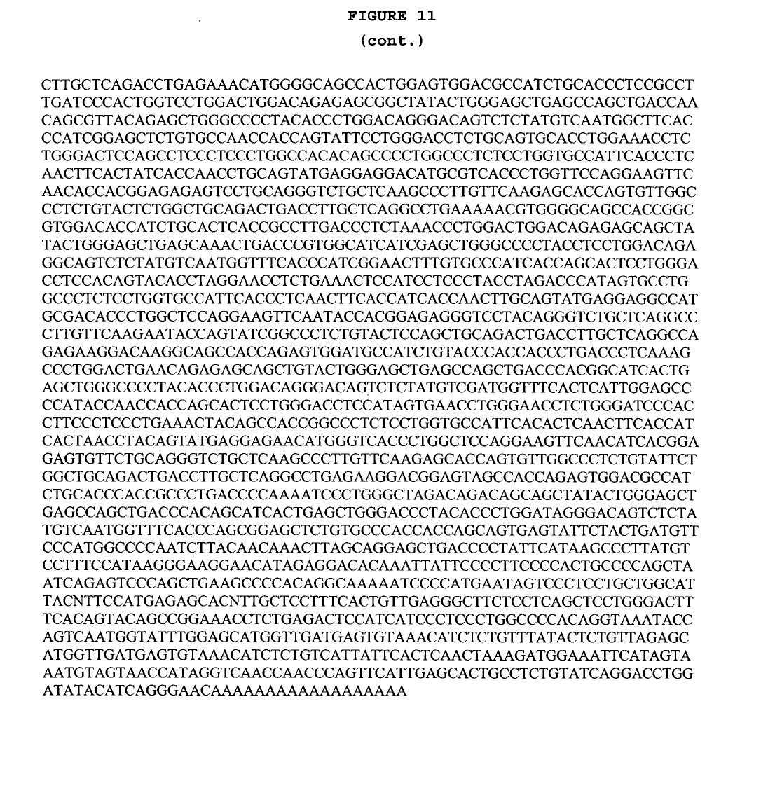

FIG. 10. Schematic showing the protein and nucleotide sequence of the 3′ end of clone B30. Also shown is the region identical to the 5′ region of clone B4. The end of repeat H and the non-translated region are shown in detail. The stop codon in the nucleotide sequence is indicated in bold type. Note that repeats A-H correspond to repeats 7-14 in FIG. 11.

FIG. 11. Nucleotide sequence of MUC16B.

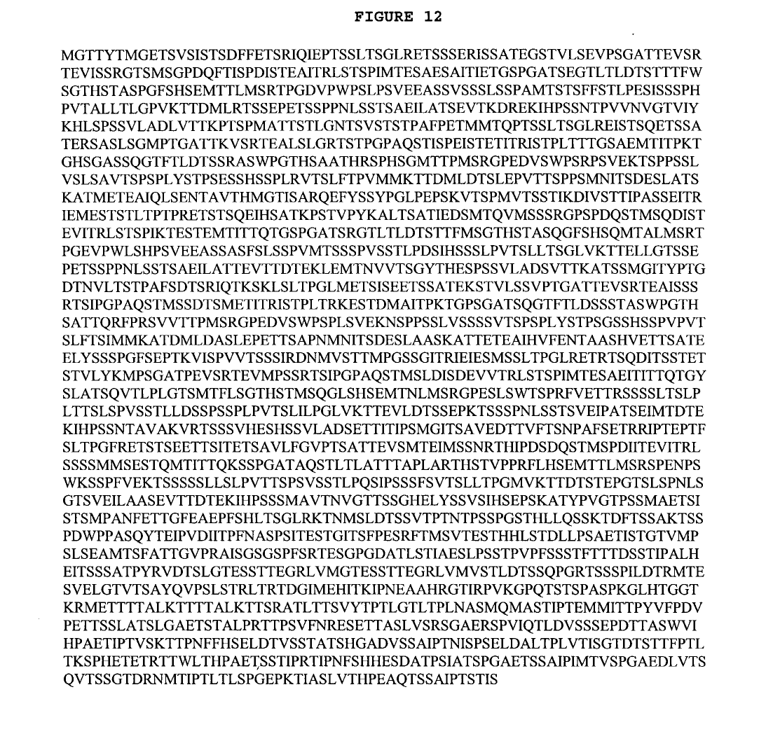

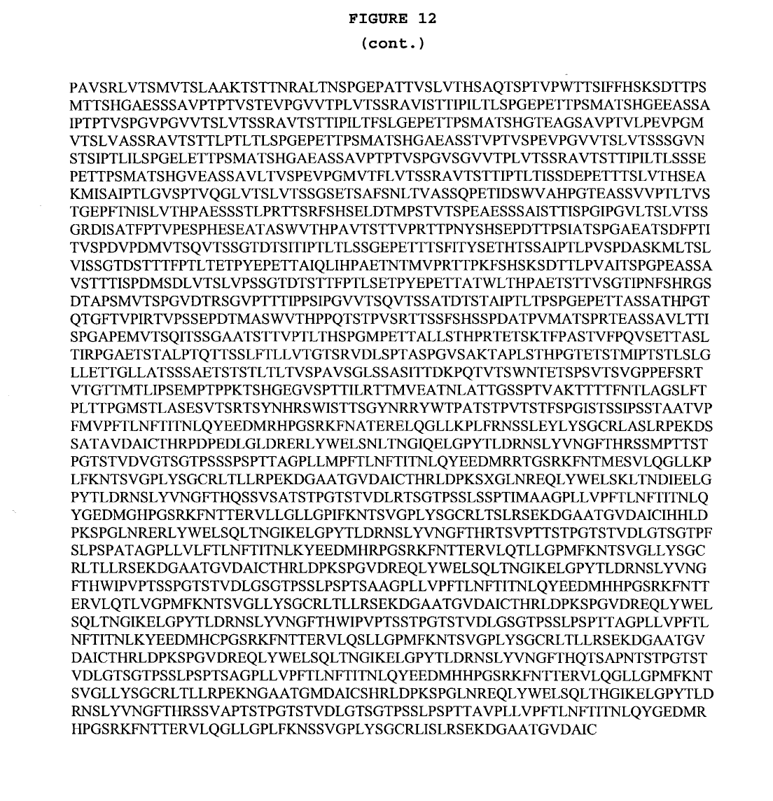

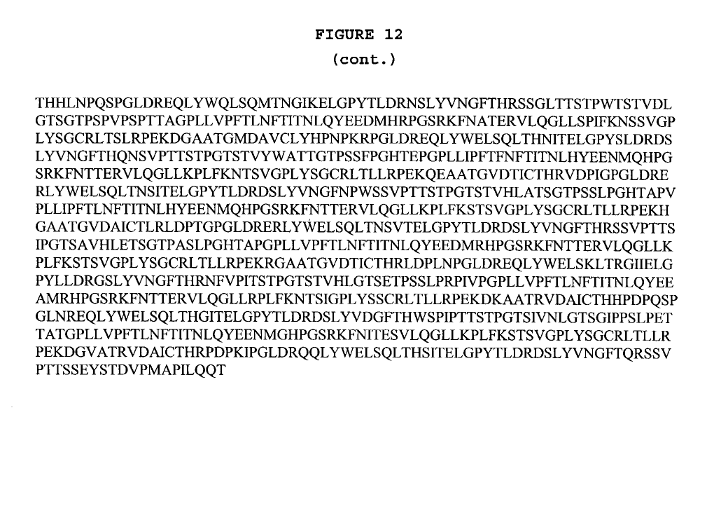

FIG. 12. Amino acid sequence of MUC16B.

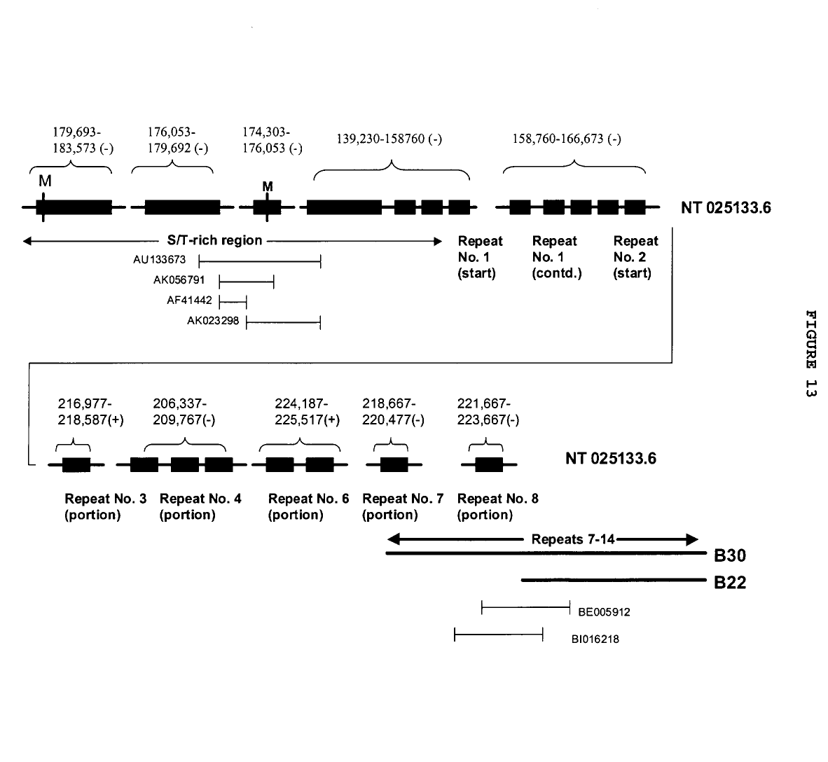

FIG. 13. Schematic showing relationship of NCBI gene sequence NT 025133.6 to clone B30 and various expressed sequence tags and the use of this information in determining the sequence of MUC16B. Exons are shown as filled boxes and the orientation of the reading frames (+ or − ) are indicated for each exon.

### DETAILED DESCRIPTION OF THE INVENTION

The invention disclosed herein provides an isolated nucleic acid molecule comprising sequences encoding the CA125 protein or a portion thereof. This invention also provides the gene encoding the CA125 protein. This invention further comprises the 5′ untranslated sequence of the CA125 gene. In addition, this invention comprises the 3′ untranslated sequence of the CA125 gene.

In addition, this invention provides the above isolated nucleic acid molecule comprising sequence set forth in FIG. 6, or a portion thereof, and the corresponding CA125 protein comprising sequence set forth in FIG. 5, or a portion thereof. Furthermore, this invention provides the above isolated nucleic acid molecule comprising sequence set forth in FIG. 7, or a portion thereof, and the corresponding CA125 protein sequence set forth in FIG. 8, or a portion thereof. In an embodiment, the nucleic acid comprises sequence set forth in FIG. 11, or a portion thereof. In another embodiment, the nucleic acid encoding protein comprises at least a portion of the amino acid sequence set forth in FIG. 12, or a portion thereof.

This invention also provides the above gene comprising sequence set forth in FIG. 10, or a portion thereof.

The invention furthermore provides the above isolated nucleic acid molecules, wherein the nucleic acid is RNA, cDNA, genomic DNA, or synthetic DNA. This invention also provides a vector comprising the above nucleic acid molecule. In an embodiment, the vector is designated as pBK-CMV-B4 comprising sequence set forth in FIG. 6, or a portion thereof, and the corresponding CA125 protein comprising sequence set forth in FIG. 5, or a portion thereof. In another embodiment, the vector is designated as pBKCMV-B30 comprising sequence set forth in FIG. 7, or a portion thereof, and the corresponding CA125 protein comprising sequence set forth in FIG. 8, or a portion thereof. In yet another embodiment, the vector is designated as pCMV-Tag-B4 comprising sequence set forth in FIG. 6, or a portion thereof, and the corresponding CA125 protein comprising sequence set forth in FIG. 5, or a portion thereof. In a further embodiment, the vector is designated as pCMV-Tag-B30 comprising sequence set forth in FIG. 7, or a portion thereof, and the corresponding CA125 protein comprising sequence set forth in FIG. 8, or a portion thereof.

This invention provides an expression system comprising the above vector. In an embodiment, the system is a eukaryotic or prokaryotic system. This invention further provides a method for producing CA125 protein comprising the above expression system.

This invention further provides an isolated nucleic acid molecule comprising sequence capable of specifically hybridizing to the sequences above. In an embodiment, the nucleic acid molecule is capable of inhibiting the expression of the CA125 protein. A method of inhibiting expression of CA125 inside a cell by vector-directed expression of a short RNA able to hybridize with the protein-coding RNA of CA125. In another embodiment, the nucleic acid molecule is at least a 7 mer. In another embodiment, it is at least a 10 mer. In a separate embodiment, the nucleic acid molecule is at least a 20 mer. In a further embodiment, the sequence is unique.

This invention further provides a method to detect ovarian cancer in a subject comprising steps of: a) contacting the above isolated nucleic acid molecule with RNA from a sample from the subject under conditions permitting the formation of a hybrid complex, and b) detecting the hybrid complex, wherein a positive detection indicates the expression of the antigen and presence of cancer.

Furthermore, this invention provides a method of monitoring ovarian cancer therapy in a subject comprising steps of: a) contacting the above isolated nucleic acid molecule with RNA from a sample from the subject under conditions permitting the formation of a hybrid complex, and b) measuring the amount of the hybrid complex, wherein a decrease in the hybrid complex indicates the success of therapy.

This invention also provides a method for inhibiting the expression of the CA125 protein comprising contacting an appropriate amount of the above nucleic acid molecule so that hybridization of the gene or transcript encoding the CA125 protein will occur, thereby inhibiting the expression of the protein. This invention further provides a composition comprising the above isolated nucleic acid molecule.

In addition, this invention provides a vaccine for a cancer which expresses CA125 protein comprising an appropriate amount of the above isolated nucleic acid molecules.

In a separate embodiment, this invention provides a vaccine for a cancer which expresses CA125 protein comprising an appropriate amount of the isolated nucleic acid molecules which, when expressed, are capable of producing a product which induces an immune response to CA125 protein. In an embodiment, the nucleic acid molecule comprises sequences encoding human CA125 protein or a portion thereof.

In another embodiment, the expressed human sequence is linked to a carrier. It is known that a carrier can booster immune response. The said carrier may be a protein carrier.

In yet another embodiment, the nucleic acid molecule comprises a nonhuman sequence. In a further embodiment, the nucleic acid molecule comprises a primate sequence. In an additional embodiment, the nucleic acid molecule comprises a murine sequence. In a further embodiment, it comprises a rat or mouse sequence. In yet another embodiment, the nucleic acid molecule comprises a synthetic sequence, which, when expressed, is capable of producing a product which induces an immune response to CA125 protein.

In addition, this invention provides the vaccine wherein the sequence hybridizes with or is homologous to the sequences encoding human CA125 protein. In an embodiment, the vaccine further comprising a suitable adjuvant. In an embodiment, the adjuvant is an alum. In another embodiment, the cancer is an ovarian, pancreatic, breast, endometrial, or lung carcinoma.

This invention also provides a method to treat a cancer which expresses CA125 in a subject comprising administering to the subject an appropriate amount of the above vaccine.

This invention also provides the above method, wherein the cancer is an ovarian, pancreatic, breast, endometrial, or lung carcinoma.

This invention further provides a vaccine for a cancer which expresses CA125 comprising an appropriate amount of the expressed CA125 protein corresponding to the above sequence.

This invention also provides a vaccine for a cancer which expresses CA125 protein comprising an appropriate amount of a substance which induces an immune response to CA125 protein. In an embodiment, the substance is a polypeptide or a peptide. In a separate embodiment, the polypeptide comprises sequences encoding human CA125 protein or a portion thereof. In yet another embodiment, the expressed human sequence is linked to a carrier. In a further embodiment, the polypeptide comprises a nonhuman sequence. In a separate embodiment, the polypeptide comprises a primate sequence. In another embodiment, the polypeptide comprises a murine sequence. In yet another embodiment, the polypeptide comprises a synthetic sequence, which, when expressed, is capable of producing a product which induces an immune response to CA125 protein. The production of a synthetic sequence or a hybrid of synthetic and natural sequences is well-known in this field. In separate embodiment, the vaccine further comprising a suitable adjuvant. In an embodiment, the adjuvant is an alum.

This invention provides the above vaccine, wherein the expressed protein is conjugated to a protein carrier to increase the immunogenicity. Furthermore, this invention provides the above vaccine, wherein the cancer is an ovarian, pancreatic, breast, endometrial, or lung carcinoma.

Furthermore, this invention provides a method to treat a cancer which expresses CA125 in a subject comprising administering to the subject an appropriate amount of the above vaccine.

This invention also provides a method to prevent a cancer which expresses CA125 in a subject comprising administering to the subject an appropriate amount of the above vaccine. In an embodiment, the cancer is an ovarian, pancreatic, breast, endometrial, or lung carcinoma.

In addition, this invention provides a method for the diagnosis of a cancer which expresses CA125 by detecting CA125-expressing cells in the blood or other fluids of patients based on the nucleic acid sequence which encodes CA125.

This invention also provides a method for monitoring the therapy of a cancer which expresses CA125 by measuring the expression of CA125-expressing cells in the blood or other fluids of patients based on the nucleic acid sequence which encodes CA125, a decrease of either the number of CA125-expressing cells or level of protein expression in the cell, indicating the success of the therapy. In an embodiment, the detection is based on polymerase chain reaction with appropriate primers.

This invention further provides a method of producing CA125 protein comprising steps of: a) constructing a vector adapted for expression in a cell which comprises the regulatory elements necessary for expression of nucleic acid in the cell operatively linked to the nucleic acid encoding the CA125 protein so as to permit expression thereof; b) placing the cells of step (a) under conditions allowing the expression of the CA125 protein; and c) recovering the CA125 protein so expressed. In an embodiment, the cell type is selected from the group consisting of bacterial cells, yeast cells, insect cells, and mammalian cells.

This invention also provides the CA125 protein expressed by the above method. This invention also provides a method for production of antibodies against CA125 protein using the protein. This invention also provides the antibodies produced by the above method. This invention also provides a method of diagnosis of cancer which expresses CA125 using the antibodies above. A method for monitoring the therapy of cancer which expresses CA125 using the above antibodies.

This invention further provides a method for determining the immunoreactive part of CA125 comprising contacting antibodies which are known to be reactive to CA125 with the protein above. Furthermore, this invention provides a transgenic nonhuman organism comprising the above isolated nucleic acid molecule. In an embodiment, the organism is a transgenic nonhuman mammal.

This invention also provides a nonhuman organism, wherein the expression of CA125 is inhibited. In an embodiment, the organism is a nonhuman mammal. In a separate embodiment, the mammal is a mouse.

Finally, this invention further provides a method for screening a compound for treatment of cancer which expresses CA125 protein comprising administering the compound to the transgenic nonhuman organism above, a decrease in expression of CA125 protein indicating that the compound may be useful for treatment of the cancer. In an embodiment, the cancer is an ovarian, pancreatic, breast, endometrial, or lung carcinoma.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

CA125 is an ovarian cancer antigen that is basis for a widely-used serum assay for the monitoring of patients with ovarian cancer, however detailed information on its biochemical and molecular nature is lacking. The inventors now report the isolation of a long, but partial, cDNA that corresponds to the CA125 antigen. A rabbit polyclonal antibody produced to purified CA125 antigen was used to screen a λZAP cDNA library from OVCAR-3 cells in Escherichia coli. The longest insert from the 53 positive isolated clones had a 5965 b.p. sequence containing a stop codon and a poly A sequence but no clear 5′ initiation sequence. The deduced amino acid sequence has many of the attributes of a mucin molecule and was designated CA125/MUC16. These features include a high serine, threonine, and proline content in an N-terminal region of nine partially conserved tandem repeats (156 amino acids each) and a C-terminal region non-tandem repeat sequence containing a possible transmembrane region and a potential tyrosine phosphorylation site. Northern blotting showed that the level of MUC16 mRNA correlated with the expression of CA125 in a panel of cell lines. The molecular cloning of CA125/MUC16 antigen will lead to a better understanding of its role in ovarian cancer.

### EXPERIMENTAL DETAILS

### First Series of Experiments

### Materials and Methods

NIH:OVCAR3 cell line was obtained from the American Type Culture Collection (Rockville, Md.). Anti-CA125 antibody mAb OC125 was a generous gift from Dr. R. Bast, Jr. mAb VK-8, developed in the inventors' Laboratory by immunization of mice with human ovarian cancer cell line OVCAR-3, also identifies CA125 but reacts with a different epitope(s) than OC125 (15). Tumor cell lines were from the Sloan-Kettering Institute Cell Bank.

### Purification of CA125 Antigen

CA125 was purified from the culture supernatant of NIH:OVCAR-3 cells in a simple two-step procedure (15). Briefly, the cells were cultured as a monolayer in a synthetic medium (ITS, Life Technologies, Grand Island, N.Y.) in RPMI medium containing 1% fetal bovine serum (FBS) and the culture medium was harvested every 7 days. Medium from 31 liters of supernatant medium was concentrated 10 fold and precipitated with perchloric acid (0.6 M final concentration). After centrifuging, the neutralized supernatant was passed through a column of normal mouse Ig-agarose (30 ml; 1.0 mg/ml) and then through a column of VK-8 mAb (80 ml; 2.0 mg/ml). The antibodies were linked to Actigel ALD gel according to the manufacturer's directions (Sterogene Bioseparations, Inc., Carlsbad, Calif.). The VK-8 column was washed at 4° with PBS, then with 1M NaCl in PBS, and finally eluted with 3M MgCl₂. Fractions (6.0 ml) were collected and assayed for CA125 antigen by ELISA with mAb VK-8 as described (15). Fractions from the MgCl₂ eluate containing CA125 reactivity were pooled and used in subsequent studies. Analysis by SDS-PAGE and silver staining (FIG. 1) showed that the sample consisted of very high molecular weight components migrating in the stacking gel and in a region just below the gel interface; all these species were reactive with mAb OC125 (data not shown). The sample also contained a lower molecular weight species originating from the FBS used in the cell cultures. The amino acid content of the sample was determined as described previously (15).

### Production of a Rabbit Antiserum to CA125 Antigen

The CA125 sample was further purified by preparative SDS-PAGE and the high molecular weight region of the gel indicated in FIG. 1 was excised. After homogenization in incomplete Freund's adjuvant the gel was used to immunize a rabbit (NZB white, female) by 3 subcutaneous injections, 1 week apart, in 8 sites. Serum was obtained from the rabbit 10 days after the final immunization. An aliquot (3.0 ml) of the serum was absorbed with a pellet of melanoma cells (SK-MEL-28, -23, -30 and -33; 6.7 ml) that had been treated with 0.2% NP40 and 0.1% protease inhibitor cocktail (Sigma Co., St. Louis, Mo.) and the absorbed serum was used to screen a cDNA library.

### Screening of OVCAR-3 cDNA Library

A cDNA library was constructed from OVCAR-3 mRNA in the λZAP Express vector in E. coli as described by the manufacturer (Stratagene, La Jolla, Calif.). The library contained 7.5×10⁶ p.f.u. The library was plated onto 15 plates at approximately 30,000 pfu/150 mm plate and plaques were transferred to nitrocellulose and screened with the absorbed rabbit antiserum (1:500). Positive plaques were identified using anti-rabbit Ig-horseradish peroxidase conjugate (Southern Biotechnology Assoc., Birmingham, Ala.) and 4-chloro-1-napthol reagent. After subcloning three times and retesting with antiserum, 54 positive clones remained. These clones contained inserts ranging from 1.5 to >4.0 kbp and were designated pBK-CMV-B1 to B54.

### DNA Sequencing and Sequence Analysis

The nucleotide sequence of the longest insert (B4) was determined using Big Dye terminators (PE Biosystems) and run on ABI 3700 or ABI 377 DNA sequencer by the Cornell University BioResource Center, Ithaca, N.Y. Using the T3 primer and then a series of internal sequencing primers, corresponding to less conserved regions of the gene, a 5965 bp sequence was identified in B4. Partial sequencing of the other inserts demonstrated that the majority corresponded to different parts of the B4 sequence.

### Northern Blot Analysis

mRNA was isolated from a panel of human tumor cell lines, which had been serologically typed for CA125 expression, using an mRNA Isolation System kit (Invitrogen, Carlsbad, Calif.). mRNA samples (3 :g) were denatured with formaldehyde, separated by electrophoresis in 1.0% agarose and transferred to nylon sheets (Gene Screen Plus, NEN, Boston, Mass.). The blot was hybridized with a biotin-labeled probe from an insert containing 3 tandem repeat regions (B53) using a chemiluminescence procedure following the manufacturer's directions (Renaissance reagent; NEN, Boston, Mass.).

### Serological Analysis

Tumor cell lines were assayed for CA125 expression with mAb OC125 and VK-8 using a red cell resetting method as described previously (15).

### RESULTS

### Cloning of CA125/MUC16 cDNA

Although most studies on the molecular cloning of mucins utilized polyclonal antisera raised to the deglycosylated mucin (apomucin), in this study we used a rabbit antiserum prepared against the native CA125 antigen. CA125 was purified by affinity chromatography on an anti-CA125 antibody (mAb VK-8) column by elution under mild conditions with a chaotropic ion (3M MgCl₂) as described previously (15). The purified sample had an amino acid composition similar to that found in other mucins (Table 1) and extremely high CA125 activity (2×10⁶ units/mg protein). To immunize rabbits the preparation was further purified by SDS-PAGE and gel slices containing high molecular weight CA125 antigen (FIG. 1) were used as the immunogen (in incomplete Freund's adjuvant). The resulting antiserum was absorbed with a pellet of non-ovarian cancer cells, after partially solubilizing the cells in 0.2% NP-40, to remove non-specific antibodies.

TABLE 1
Comparison of Amino Acid Content of Purified CA125 and Deduced
Amino Acid Composition of CA125/MUC16 and Its Tandem Repeat
Region

| | Purified | CA125/ | CA125/ |
|---|---|---|---|
| | CA125 | MUC16 | MUC16 (TR) |
| Amino Acid | moles % | moles % | moles % |
| Asn | 8.5 | 8.9 | 8.1 |
| Glx | 7.8 | 8.1 | 7.5 |
| Ser | 11.0 | 8.7 | 8.9 |
| Gly | 9.0 | 7.4 | 7.6 |
| His | 2.6 | 2.8 | 2.9 |
| Arg | 4.6 | 5.9 | 6.3 |
| Thr | 12.4 | 11.6 | 12.7 |
| Ala | 3.8 | 3.1 | 2.9 |
| Pro | 8.7 | 8.1 | 9.0 |
| Tyr | 2.6 | 3.8 | 3.3 |
| Val | 5.2 | 5.0 | 4.7 |
| Met | 1.2 | 1.1 | 1.0 |
| Cys | — | 1.4 | 1.2 |
| Iso | 2.7 | 3.3 | 3.1 |
| Leu | 12.4 | 13.4 | 13.7 |
| Phe | 3.7 | 3.9 | 3.6 |
| Lys | 3.8 | 3.0 | 2.9 |

The absorbed antiserum was used to screen a λZAP cDNA library from OVCAR-3 cells expressed in E. coli. Fifty-four positive clones were detected and 53 inserts were sequenced. Initial sequencing of the longest clone (B4) showed that it had 9 partially conserved repeats of 495 b.p. each and a short non-repetitive 3′ region. Further sequencing with internal primers extended the 3′ end of the sequence to include a stop codon, a polyadenylation signal and a poly A region for a total of 5965 b.p. (FIG. 2). No clear initiation sequence (ATG in a Kozak box) was detected at the 5′-end, indicating that the derived sequence is incomplete. The majority of the other inserts (B1-B53) had sequences derived from different parts of the B4 sequence. No clones containing only 3′ non-repetitive sequences were identified. Searching GenBank™ revealed no related full-length cDNA but numerous related human ESTs (including Accession Numbers: AI566650, AI537678, AI276341, AI923224, AI276341, AU158364, AU140211, AK024365) and one mouse EST (AK003577) were detected. With minor exceptions, these sequences were identical to those derived for B4. The nucleotide sequence of B4 was designated CA125/MUC16.

### Chromosomal Location of CA125/MUC13 Sequences

Comparison of the B4 sequence with the working draft version of the human genome, available from the NCBI, located homologous sequences on chromosome 19 (p13.3 region). As sequencing of this region is incomplete and presently consists of numerous unordered segments of varying lengths, more complete genomic information must await the availability of further sequencing data.

### Analysis of the Deduced Amino Acid Sequence of CA125/MUC16

The nucleotide was conceptually translated into an amino acid sequence assuming initiation at the ATG of the β-galactosidase gene in the vector. The deduced amino acid sequence of 1890 amino acids (FIG. 3) suggested a mucin-type molecule. It had an amino acid composition that was moderately high in serine (8.9%), threonine (12.5%) and proline (8.8%); this composition is very similar to that of the purified CA125 sample used in this study (Table 1), although the proportion of these three amino acids is lower than in most other mucins. The sequence contained a large region of 9 tandem repeats (TR) of 165 amino acids each and a C-terminal non-repetitive region of 537 amino acids. None of the 9 repeats are identical but numerous perfectly conserved residues and short sequences are apparent

(FIG. 3). Two conserved cysteine residues within the TRs are notable. The serine and threonine residues are scattered throughout the sequence but the TR regions have prominent clusters of Ser and Thr, often with adjacent Pro residues which is a common feature of O-glycosylation sites (19), e.g. SSVPTTSTP (47-55 and 671-679) and SSVSTTSTTSTP (1139-1147). These characteristics are typical of mucins. The high Leu content of this sequence is, however, not found in other cloned mucins. Other features of interest include a sequence of hydrophobic amino acids (25 residues) towards the C-terminal end (presumably representing a transmembrane region) and a short 31-amino-acid cytoplasmic tail. This region also contains a consensus tyrosine phosphorylation site (RRKKEGEY; refs. 20, 21). Numerous potential N-linked glycosylation sites occur in both the TR and non-TR regions (FIG. 3).

### Northern Blotting

mRNA from a panel of ten CA125⁺ and CA125⁻ cell lines was screened with a probe derived from the tandem repeat region of MUC16. Three of the cell lines gave positive blots and 7 were unreactive (FIG. 4). The polydisperse pattern obtained is typical of that observed with other mucin mRNAs. These data corresponded to the expression of CA125 antigen on the cell lines as determined by serological analysis with antibodies to CA125 (mAbs OC125 and VK-8). The strongest signal was given by mRNA from OVCAR-3 (lane 5), the cell line from which the CA125 was purified and the cDNA library was produced.

### Peptide Sequences Derived from CA125 Antigen

Purified CA125 was deglycosylated by treatment with anhydrous HF at room temperature for 3 hrs (22). Two sequences were obtained from a tryptic digest of the HF-treated sample after SDS-PAGE and transfer of the 25-35 kDa region to a nitrocellulose membrane (22). The product was also digested with Lys-C in guanidinium hydrochloride; peptides were isolated by microbore HPLC, and four peptides were successfully sequenced (Table 2). Five of these peptides corresponded to sequences within the TR and one to a sequence in the C-terminal region of the deduced MUC16 sequence (Table 2).

TABLE 2
Amino Acid Sequences Derived from Purified CA125

| | Position in |
|---|---|
| Sequence | CA125/MUC16 sequences |
| By Lys-C digestion | |
| AQPGTTNYQRNK | 1722-1733 |
| SPRLDR | 1098-1113 |
| PLFK | 120-123, and other locations |
| PGL | 7-9 and other locations |
| By trypsin digestion | |
| KAQPGTTNYQRN | 1721-1732 |
| RTPDTSTMHLATSRT | 833-847 |

### EXPRESSION ANALYSIS OF CA125 NUCLEOTIDE CLONE (FIG. 9)

This figure is the result of an expression experiment that confirms that the sequence actually codes for CA125, as recognized by standard antibodies.

### Method

Clone B53 (in pCMV-tag vector) was transfected into SK-OV-3 (CA125-negative cell line) with Lipofectamine Plus reagent. Stable clones were selected with neomycin. Cells were radiolabeled with ³H glucosamine, immunoprecipitated with antibodies and the products analyzed by SDS-PAGE and autoradiography.

### Result

Lane 1 (mAb OC125) and lane 2 (mAb VK-8) have bands at the top of the gel showing the presence of CA125 antigen in the transfected cells. No bands were obtained with normal mouse serum (negative control).

This result proves that the cloned nucleotide sequence contains the information for coding for the CA125 antigen.

### DISCUSSION

Based on the following evidence, the cloned MUC16 sequence is a strong candidate for being the cDNA for the peptide core of the CA125 antigen: (i) the CA125 antigen used in the study was isolated by affinity chromatography on an anti-CA125 monoclonal antibody column and was highly purified, (ii) peptides isolated from the purified CA125 sample corresponded to sequences in the cloned MUC16 sequence and (iii) MUC16 mRNA levels in a panel of cancer cell lines, as determined by Northern blotting, correlated with the expression of CA125 in the cell lines as determined serologically. Moreover, this result supports earlier biochemical studies that had concluded that CA125 antigen is a mucin-type molecule (15). The cloned sequence is therefore designated as CA125/MUC16. This gene has been provisionally localized to chromosome 19p13.3. Initially reported sequences of mucins are rarely full length because of the extremely large size of mucin mRNAs and not unexpectedly, no apparent 5′ initiation signal is evident in the CA125/MUC16 cDNA sequence. The sequence is believed to be complete at the 3′-end as a stop codon, a polyadenylation site and a poly A tail have been identified (FIG. 2).

Mucins are notoriously difficult to clone because of their complex structure and high degree of glycosylation. Most successful cloning efforts have resulted from screening cDNA libraries with a polyclonal antiserum produced to the deglycosylated mucin (reviewed in 23-27). Thirteen human mucins have been cloned or partially cloned to date (MUC-1, -2, -3, -4, -5AC, -5B, -6, -7, -8, -9, -11, -12 and -13; refs. 23-29). In this study, however, a polyclonal antiserum to the native mucin was used to isolate a cDNA corresponding to the peptide moiety of CA125/MUC16 antigen. This approach may have been successful because of the relatively low content of serine and threonine (representing potential O-glycosylation sites) in CA125/MUC16 in comparison with most other mucins. The high degree of purity of the isolated antigen, as well as the use of a highly absorbed antiserum and the high expression of CA125 in the OVCAR-3 cell line used to produce the cDNA library, may also have been key factors in obtaining positive clones.

The deduced amino acid sequence of CA125/MUC16 resembles other mucins in having serine, threonine and proline as major amino acids; however, its high content of leucine is characteristic of MUC16. The presence of tandem repeats is also typical of mucins but the length of the repeat units (156 amino acids) is unusual, with only MUC6 having longer tandem repeats (30). Nine TRs have been identified thus far, with the last repeat being shorter than the others. The amino acid sequences in the TRs are not perfectly conserved, although 81 positions have conserved amino acids and certain motifs e.g. GPLYSCRLTLLR, ELGPYTL, FTLNFTIXNL and PGSRKFNXT, are found in all or most of the TRs. Two closely spaced cysteine residues (20 amino acids apart), which could form interchain disulfide bonded loops in the structure, are also perfectly conserved.

Serine and threonine residues, representing potential O-glycosylation sites, are scattered throughout the sequence but blocks of clustered Ser and Thr residues are evident in the TR region. These regions have adjacent or nearby Pro residues—a motif that is frequently found in O-glycosylation sites (19). One short serine/threonine-rich sequence (PTSSSST) is also found in the C-terminal non-TR region. Numerous potential N-glycosylation sites (Asn-X-Ser/Thr, where X is any amino acid except Pro) are also found in the sequence, including two that are perfectly conserved in the TR region. It is unlikely, however, that many of these sites are used as the content of N-linked glycan chains in purified CA125 is very low (15). It is also interesting to note that the sequence contains numerous lysine and arginine residues that are remote from the postulated O-glycosylation sites and which could explain the sensitivity of CA125 to trypsin digestion (16). Searching for conserved domains in the NCBI Blast site revealed the presence of six SEA domains in the deduced protein sequence. The significance of this finding is unclear. Five of the domains are in the tandem repeat region and one is in the non-tandem repeat region (amino acids 1709-1768). SEA domains were originally described as being characteristic of membrane-bound proteins with high levels of O-glycosylation (31); CA125/MUC16 certainly fits this description. Recently, it has been suggested that they also designate regions susceptible to proteolytic cleavage (32).

Two features of the non-TR region are particularly interesting. First, is the presence of a 25-amino-acid block of hydrophobic amino acids which could represent a membrane-spanning region. Transmembrane (TM) motifs have been found in five other mucins (MUC-1, -3, -4, -12 and 13). The remainder of the mucins that have been cloned lack TM regions and instead have cysteine-rich regions with homology to van Willebrand factor (27). Members of this family of mucins are secreted and form gels that protect and lubricate epithelial tissues. CA125 is also secreted from ovarian tumors and cell lines but the mechanism for its secretion is unclear. Two possibilities can be suggested—(i) a proteolytic event, possibly in the C-terminal SEA domain, cleaves off the luminal N-terminal domain (as in MUC1, refs. 33, 34) or (ii) alternatively-spliced mRNAs are generated that lack the TM region. Indeed, recent sequencing of clones B30 and B22 indicates the existence of such sequences (data not shown). The second feature of interest in the non-TR sequence is a short cytoplasmic tail (31 amino acid) that contains a putative tyrosine phosphorylation site (RRKKEGEY). This sequence is conserved in the translated mouse EST (AK003577) that has homology with CA125/MUC16 at the C-terminal end. MUC-1 has several tyrosine residues in its cytoplasmic tail and at least one of these is phosphorylated in vivo (35, 36). One of the Tyr residues in MUC1 occurs in a YTNP sequence, a motif that is responsible for binding to SH2 domains in proteins involved in intracellular signaling. The putative phosphorylation site found in CA125/MUC16 was first recognized in src family proteins (19, 20). Whether or not this tyrosine residue is phosphorylated in CA125 antigen is not known. Fendrick et al. (37) reported the presence of phosphate in CA125 from WISH cells by labeling with ³²PO₄⁼ and immunoprecipitation analysis but concluded that the phosphorylation site(s) are on Ser or Thr. Significantly, however, the secretion of CA125 is stimulated by epidermal growth factor (EGF), presumably through the EGF receptor which is a well-known tyrosine kinase (37). The possibility that CA125/MUC16 is phosphorylated on tyrosine and is involved in intracellular signaling needs further investigation. Interestingly, no EGF domains, which are found in some other mucins (MUC3, MUC4, MUC12 and 13), were located in CA125 (MUC16).

The molecular cloning of CA125 antigen opens the way to a better understanding of this important antigen, including its physiological function and its role in the biology of ovarian cancer. Of immediate interest will be the identification of the epitope(s) recognized by the various monoclonal antibodies that recognize CA125 (38). The identification of tandem repeats in the MUC16/CA125 structure is consistent with the use of a single monoclonal antibody in double-determinant assays for CA125 levels, which would indicate that the antigen has multiple, identical epitopes (2). Such studies could lead to improvements in the CA125 assay for the detection of ovarian cancer.

### REFERENCES

1. Bast, R. C., Jr., Feeney, M., Lazarus, H., Nadler, L. M., Colvin, R. C. and Knapp, R. C. (1981) J. Clin. Invest. 68, 1331-1337

2. Bast, R. C., Jr., Klug, T. L., St John, E., Jenison, E., Niloff, J. M., Lazarus, H., Berkowitz, R. S., Leavitt, T., Griffiths, C. T., and Parker, L., et al. (1983) N. Engl. J. Med. 309, 883-887

3. Bast, R. C., Jr., Xu, F. -J., Yu, Y. H., Barnhill, S., Zhang, Z., and Mills, G. B. (1998) Int. J. Biol. Markers 13, 179-187

4. Verheijen, R. H., Von Mensdorff-Pouilly, S., Van Kamp, G. J., and Kenemans, P. (1999) Sem. Cancer Biol. 9, 117-124

5. Menon, U. and Jacobs, I. J. (2000) Curr. Opin. Obstet. Gynecol. 12, 39-42

6. Meyer, T. and Rustin, G. J. (2000) Br. J. Cancer 82, 1535-1538

7. Meden, H. and Fattahi-Meibodi, A. (1998) Int. J. Biol. Markers 13, 231-237

8. O'Brien, T. J. (1998) Int. J. Biol. Markers 13, 188-195

9. Davis, H. M., Zurawski, V. R., Bast, R. C., Jr., and Klug, T. L. (1986) Cancer Res. 46, 6143-6148

10. Matsuoka, Y., Nakashima, T., Endo, K., Yoshida, T., Kunimatsu, M., Sakahara, H., Koizumi, M., Nakagawa, T., Yamaguchi, N. and Torizuka, K. (1987) Cancer Res. 47, 6335-6340

11. Nagata, A., Hirota, N., Sakai, T., Fujimoto, M., and Komoda, T. (1991) Tumour Biol. 12, 279-286

12. de los Frailes, M. T., Stark, S., Jaeger, W., Hoerauf, A., and Wildt, L. (1993) Tumour Biol. 14, 18-29

13. Kobayashi, H., Ida, W., Terao, T., and Kawashima, Y. (1993) Am. J. Obstet. Gynecol. 169, 725-730

14. Zurawski, V. R., Jr., Davis, H. M., Finkler, N. J., Harrison, C. L., Bast, R. C., Jr., and Knapp, R. C. (1988) Cancer Rev. 11-12, 102-118

15. Lloyd, K. O., Yin, B. W. T., and Kudryashov, V. (1997) Int. J. Cancer 71, 842-850

16. Lloyd, K. O. and Yin, B. W. T. (2001) Tumor Biol. 22, 77-82

17. Campbell, I. G., Campbell, I. G.,., Foulkes, W. D., Senger, G., Stamp, G. W., Allan, G., Boyers, C., Jones, K., Bast, R. C., Jr., and Solomon, E. (1994) Hum. Mol. Gen. 3, 589-594

18. Chambers, J. A. and Solomon, E. (1996) Genomics 38, 305-313

19. Hansen, J. E., Lund, O., Engelbrecht, J., Bohr, H., Nielsen, J. O., Hansen, J. -E. S., and Brunak, S. (1995) Biochem. J. 308, 801-813

20. Patschinsky, T., Hunter, T., Esch, F. S., and Cooper, J. A. (1982) Proc. Natl. Acad. Sci. USA 79, 973-977

21. Cooper, J. A., Esch, F. S., Taylor, S. S., and Hunter, T. (1984) J. Biol. Chem 259, 7835-7841

22. Lloyd, K. O., Yin, B. W. T., Tempst, P., and Erdjument-Bromage, H. (2000) Biochim. Biophys. Acta Gen. Subj. 1474, 410-414

23. Taylor-Papadimitriou, J. and Gendler, S. J. (1988) Cancer Rev. 11-12, 11-24.

24. Kim, Y. S., Gum, J. R., Jr., Byrd, J. C., and Toribara, N. W. (1991) Am. Rev. Respir. Dis. 144 Suppl., S10-S14

25. Gendler, S. J. and Spicer, A. P. (1995) Annu. Rev. Physiol. 57, 607-634

26. Seregni, E., Botti, C., Massaron, S., Lombardo, C., Capobianco, A., Bogni, A., and Bombardier, E. (1997) Tumori 83, 625-632

27. Perez-Vilar, J. and Hill, R. L. (1999) J. Biol. Chem. 274, 31751-31754

28. Williams, S. J., McGuckin, M. A., Gotley, D. C., Eyre, H. J., Sutherland, G. R., and Antalis, T. M. (1999) Cancer Res. 16, 4083-4089.

29. Williams, S. J., Wreschner, D. H., Tran, M., Eyre, H. J., Sutherland, G. R., and McGuckin, M. A. (2001) J. Biol. Chem.—in press

30. Toribara, N. W., Roberton, A. M., Ho, S. B., Kuo, W. -L., Gum, E., Hicks, J. W., Gum, J. R., Jr., Byrd, J. C., Siddiki, B., and Kim, Y. S. (1993) J. Biol. Chem. 268, 5879-5885

31. Bork, P. and Patthy, L. (1995) Protein Sci. 49, 1421-1425.

32. Wreischner, D. H., Keydar, I., Yoeli, M., Okun, L., Ziv, R., William, S., and McGuckin (2000). Proc. 6^{th} Int. Workshop on Carcinoma-associated Mucins, Cambridge, UK. p. 25.

33. Ligtenberg, M. J., Kruijshaar, L., Buijs, F., van Meijer, M., Litvinov, S. V., and Hilkens, J. (1992) J. Biol. Chem 267, 6171-6177

34. Boshell, M., Lalani, E. -N., Pemberton, L., Burchell, J., Gendler, S., and Taylor-Papadimitriou, J. (1992) Biochem. Biophys. Res. Commun. 185, 1-8

35. Zrihan-Licht, S., Baruch, A., Elroy-Stein, O., Keydar, I., and Wreschner, D. H. (1994) FEBS Lett. 356, 130-136

36. Pandey, P., Kharbanda, S., and Kufe, D. (1995) Cancer Res. 55, 4000-4003

37. Fendrick, J. L., Konishi, I., Geary, S. M., Parmley, T. H., Quirk, J. G., Jr., and O'Brien, T. J. (1997) Tumour Biol. 18, 278-289

38. Nustad, K., Bast, R. C., Jr., O'Brien, T. J., Nilsson, O., Seguin, P., Suresh, M. R., Saga, T., Nozawa, S., Bermer, O. P., and de Bruijn, H. W. A., Nap, M., Vitali, A., Gadnell, M., Clark, J., Shigemasa, K., Karlsson, B., Kreutz, F. T., Jette D., Sakahara, H., Endo, K., Paus, E., Warren, D., Hammarstrom, S., Kenemans, P., and Hilgers, J. (1996) Tumour Biol. 17, 196-219

### Second Series Of Experiments

### Identification of a Form of the CA125 Ovarian Cancer Antigen (MUC16B) Lacking a Transmembrane Sequence

CA125 antigen is overexpressed in the majority of human ovarian carcinomas and is released into the blood stream where it can be detected with suitable immunological assays (1). Approximately 80% of patients with ovarian cancer have elevated serum CA125 levels and the measurement of these levels is a valuable tool for monitoring the clinical status of ovarian cancer patients (2,3).

Despite the widespread use of CA125 as a serum marker, until recently, very little information was available on the molecular nature of the CA125 antigen. Biochemical studies had indicated that the antigen is a large, highly glycosylated glycoprotein with mucin-like characteristics (4-6). This suggestion has now been confirmed by the molecular cloning of CA125 (gene designation: MUC16) by the inventors (7,8) and O'Brien and coworkers (9). Both groups reported a long DNA species that coded for a protein with a large number of partially-conserved, 156 amino acid-long tandem repeat (TR) sequences. These tandem repeats contain a serine, threonine and proline-rich (S/T-rich) area that is a potential region of O-glycosylation. The molecule also contains a C-terminal non-TR region, a potential membrane-spanning sequence and a short cytoplasmic tail. O'Brien et al. (9) also reported a large N-terminal non-repetitive S/T/P-rich region in CA125.

The presence of a membrane-spanning region in MUC16/CA125 raises the question as to the source of serum CA125 antigen. One possibility is that cell-bound CA125 is cleaved by a protease(s) and released into the surrounding medium. In support of this mechanism is the presence in the molecule of SEA motifs which are possible protease-sensitive sites (7,9). Another, not mutually exclusive, explanation is that MUC16/CA125 is also synthesised as a form lacking a transmembrane region that could be directly secreted from cells.

During the original cloning of MUC16/CA125 we had isolated a small number of cDNA clones that appeared to differ from the reported clone (B4) in having a different 3′ nucleotide sequence. We now show that these species represent a second form of MUC16/CA125 lacking a C-terminal membrane-spanning region that could be a secreted form of the antigen. This species (gene designation: MUC16B) also has a long serine/threonine-rich N-terminal sequence.

### EXPERIMENTAL PROCEDURES

### Materials and Methods

The isolation of cDNA clones B4, B30 and B22 in the pBK-CMV vector has been described (7). Human tumor cell lines OVCAR3, SK-OV-8, COLO316, 2774, SK-OV-3 and SK-OV-8 (ovarian cancer cell lines), MCF-7 (breast cancer), IMR-32 (neuroblastoma), MKN45 (gastric cancer), and MCA (sarcoma) and their CA125 status have been described (7).

### RT-PCR Procedure and cDNA Sequencing

Messenger RNA was isolated from cell pellets using a FastTrack 2.0 kit (Invitrogen Life Technologies, Carlsbad, Calif.). cDNA was then synthesised using a Superscript First Strand Synthesis kit as described by the manufacturer (Invitrogen). RT-PCR was performed as follows: 2 μl cDNA, 0.2 mM dNTP mix, 4 mM MgCl2, 0.4 to 1 μM forward or reverse primers and 2.5U Platinum Taq DNA Polymerae (Invitrogen) were mixed in a total volume of 50 μl and the samples were cycled as follows: 94° for 1 min., 25-35 cycles of 94° C. for 30 secs, 54-65° C. for 30 secs and 72° C. for 30 secs to 3 min. and a final cycle of 94° C. fro 5 min. For the PCR of longer products (>5 kb) the LA PCR kit from Takara Sfuko Co. was used under following conditions: 94° C. for 1 min., followed by 30 cycles of 94° C. for 20 secs., 60° C. for 30 secs and 72° C. for 7 Or 10 min. and a final cycle of 94° C. for 20 secs., 55 or 60° C. for 30 secs., and 72° C. for 10 min. RT-PCR products were analyzed by gel electrophoresis in 0.8 or 1.0% agarose in Tris-acetate-EDTA and stained with ethidium bromide.

For sequencing the PCR product was cloned into the Topo TA cloning vector from Invitrogen). Inserts were sequenced initially with T3 and T7 primers and then with suitable forward and reverse primers designed according to the derived sequence. Sequencing was performed either by our own sequencing facility or by the Cornell University Facility using a BigDye Terminator Primer Sequencing Kit (Perkin Elmer/ABI) in ABI 3700 or ABI 377 DNA seqenators. The sequences were aligned visually for the repeat region sequences and with the aid of Vector NT for other sequences.

### 3′ and 5′ RACE Procedures

These procedures were performed with the First Choice RLM-RACE kit (Ambion Co., Austin Tex.) using suitable forward primers for the 3′ and reverse primers for 5′ region respectively. For the 5′ RACE the outer gene-specific primer was 5′TCACAGTCCCTACATTGACTA3′ and the inner primer was 5′CATGGCACATCTCCAGGGT3′. The products' were cloned into TA vector and sequenced as described above.

### RESULTS

### Cloning and Sequencing of B30 cDNA

During the original expression cloning of MUC16 (7) we observed that the majority of the clones detected by screening the cDNA library with a rabbit antiserum were shorter forms of the longest clone (B4) reported (7) and contained varying numbers of TRs, a non-TR-region, a potential TM region and a cytoplasmic tail. However a few clones were isolated that appeared to be different in that they lacked a restriction enzyme site (Xho) present in the B4 family of inserts. The cDNA from one of these clones (B30) was completely sequenced using the T3 primer of the vector initially and, subsequently, new forward and reverse primers derived from the less conserved regions of the new sequence. The B30 insert had a total of 4103 bp with a stop codon at 3593 bp. This was followed by 3′ non-translated region and finally, a poly A sequence. Despite the presence of a poly-A sequence no obvious polyadenylation site was observed (FIG. 10). Clone B22 was partially sequenced and shown to be a shorter (2432 bp) form identical to the 3′ sequence of B30.

Conceptual translation of the B30 sequence indicated a protein composed entirely of 7.7 TRs of 156 amino acids each. The 4.5 C-terminal repeats were identical to sequences found in the B4 clone and three new partially-conserved TRs were detected N-terminal to the B4 sequence. The new repeats contained the potential cysteine loop, the 2 conserved N-glycosylation sites and the serine/threonine-rich region found in clone B4 of MUC16. No non-TR, transmembrane or cytoplasmic sequences were present in this new species of MUC16. Searching the NCBI database with this sequence yielded two EST (BE005912 and BI016218) corresponding to repeat number 3 in the B30 sequence. Surprisingly, no ESTs, or even genomic, sequences corresponding to the non-translated 3′ region of B30 were detected in the NCBI databases. In order to confirm that the new form of MUC16 was not a cloning artifact 3′ RACE was performed with RNA from the OVCAR3 cell line. Sequences corresponding to the last repeat and the untranslated region were identified (data not shown). We also examined a panel of cancer cells for transcripts corresponding to the 3′ region by RT-PCR using primers from repeat 8 and the 3′ end of the untranslated region of B30. PCR products were found only with mRNA from cells known to express CA125, again confirming the relationship of B30 to CA125.

### Complete Sequence of MUC16B/CA125

Searching the NCBI genomic database with sequences derived from B30 indicated that numerous sequences related to this species were located on a genomic sequence file designated NT 025133.6 (FIG. 13). At present (March 2002), this region, located on chromosome 19 p13.3/p13.2, consists of 53 unordered sequences of varying length. This data does not allow the complete sequence of MUC16 to be easily assembled, however by designing suitable RT-PCR primers from the genomic sequence for RT-PCR it was possible to amplify and sequence cDNA that extended the B30 by 6.5 partially conserved tandem repeat units (FIGS. 11 and 12). This results in the identification of a total of 14 repeats in the new MUC16 sequence. Adjacent to the first exon of the 5′-most repeat sequence in NT 025133.6 we noticed a very long potential open reading frame. This region does not contain any repeat sequences but is rich in serine, threonine and proline residues. Also, in NT 025133.6 we observed a short putative exon containing the ATG sequence suggested by O'Brien et al. (9) to be the initiating codon of CA125 (FIG. 13). Again by designing suitable primers in this region, PCR products corresponding to this new 5′ region were cloned and sequenced. The NCBI database contains ESTs corresponding to portions of the 5′ region of this sequence (AK056791, AK056791 and AF41442). One of these ESTs extended into the 5′ region beyond the ATG designated by O'Brien et al. (9). In fact NT 025133.6 contains an extremely long potential open reading frame (positions 176,04,53-179,693) corresponding to this region. The Celera public access database also contains genomic sequence for this region and, significantly, has an extremely long hypothetical transcript sequence (hCT1645865) containing all the putative exons in 176,053-179,693 and 139,330-158,760 b.p. regions of NT 025133.6. Primers were also designed to sequence these regions and by application of RT-PCR to OVCAR-3 mRNA it was possible to confirm these sequences. Only minor differences between the experimentally-derived sequence and the data base sequences except for numerous differences in the 3′ region of the serine/threonine-rich were it joins the tandem repeat region between the published data and our sequence. This long S/T/P-rich coding region has numerous ATG codons which could serve as initiation sites for mRNA synthesis (some of them fitting a Kozak consensus motif, ref. 10) was difficult to pick a likely site. Application of 5′ RACE with a series of primers in different locations in the sequence finally yielded a primer that gave a clear cDNA product and sequencing of this product indicated a start site at position 261 (FIGS. 11 and 12). This ATG is located in a classical Kozak box. To confirm that the 5′ S/T/P-coding region was in fact related to the tandem repeat region and codes for the CA125 antigen we performed RT-PCR on mRNA from a panel of cell lines (as we had done for the 3′ end) with primers corresponding to a sequence close to the 5′ end; the result showed a complete correlation between generation of the bp product and expression of CA125 in these cell lines.

Conceptual translation of the assembled nucleotide sequence (18405 bp) demonstrated a protein of 5851 amino acids with an extremely long (3650 amino acids) S/T/P-rich C-terminal (containing 17.2% serine, 19.5% threonine and 9.0% proline) followed by a region of 14 partially-conserved repeats of 156 amino acids each as described above (FIG. 12). The sequence terminated after one of the S/T/P-rich regions in the last TR with no hydrophobic C-terminal transmembrane region being observed.

### DISCUSSION

Using a combination of expression cloning and RT-PCR approaches we have identified a new species of CA125 (designated MUC16B) that has a long serine/threonine-rich N-terminal region and a C-terminal region of 14 tandem repeats but no apparent transmembrane region. This product could therefore be a secreted form of CA125 although no secretory peptide sequence is present at the N-terminus. The tandem repeat region is similar in construction to the repeats previously observed in MUC16/CA125. These repeats contain a small region rich in serine and threonine which could represent O-glycosylation sites. The N-terminal region has numerous serine and threonine residues scattered through the sequence and these could also be O-glycosylated. CA125 is known to be highly glycosylated (77% by weight) and most of this consists of O-glycosylated chains (4). Two conserved potential N-glycosylated sites occur in each tandem repeat and these could also contribute to the carbohydrate content of CA125, although this level is probably quite low (4).

At present it is unclear as to whether the CA125 molecules identified by the inventors (7,8) and O'Brien et al. (9) have the same long N-terminal sequence. O'Brien et al. (9) described a N-terminal sequence of 1638 amino acids in contrast to the xxx amino acids described here for MUC16B. However, the S/T/P-rich region was connected to the TR regions and the non-TR, trans-membrane and cytoplasmic regions similar to those reported by us in MUC16/CA125. Using 5′ RACE they detected an initiating methionine (at position 6435 in FIG. 11) whereas we could detect such a site only at position 262. Also unclear is whether either of the N-terminal S/T/P-rich sequences are present in the MUC16/CA125 species reported previously as clone B4 was not complete at the 5′ end (7). We were unable to generate products by performing RT-PCR with primers located in MUC16B repeat region and in the 3′ portion of the MUC16 tandem repeats not found in MUC16B, indicating that MUC16 and MUC16B have different repeat sequences at their 5′-end and possibly, therefore, a shorter or different S/T-rich regions.. Such a situation may account for the larger number of repeats that were identified by O'Brien et al. (9) and those that can be found in the genome data bases and not in MUC16B.

MUC16B/CA125 is an extremely long molecule with a peptide chain of 5851 amino acids and an Mr of about 600,000. Many other cloned mucins (11,12) also have extremely long peptide sequences, e. g. MUC5B has 5662 amino acids and a Mr of about 600,000 (13). By pulse-chase experiments we had previously identified a putative CA125 precursor species of about 400 kDa which, given the uncertainties inherent in very high molecular sizes determined by SDS-PAGE, is consistent with this result (5). It is also interesting to note that the precursor consisted of a doublet of two closely-spaced species on SDS-PAGE which could correspond to MUC16 and MUC16B (5).

Although MUC16B/CA125 has many of the attributes expected of a mucin species (i.e. large size, high serine, threonine and proline content, high level of O-glycosylation and presence of tandem repeats) it also has some unique features. These include the presence of potential cysteine loops in the repeat region and the segregation of the O-glycosylation sites into a small region of each repeat. Another unusual feature is that the repeat region is not coded by one long exon; rather each repeat unit contains 5 small exons [O'Brien et al. (9) and our unreported data]. In CA125 the longest exons are found at the 5′ end and code for a non-repeat serine/threonine-rich region. Because of it large size CA125 is extremely difficult to isolate in an intact form from biological materials. In our original purification of CA125 we described an extremely large species migrating in the stacking gel of a SDS-PAGE gel (4), whereas subsequently we found smaller species migrating mainly in the upper region of the separating gel (7). Recently, in a report from the Third ISOBM Workshop (14) it was reported that CA125 can be degraded by sonication procedures, as well as by proteolytic digestion.

Another feature of CA125 that still needs to be completely elucidated is the location in the molecule of the antibody-detected epitopes. Presently available data indicated that they are mainly located in the tandem repeat regions of the molecule (8,9)and this would be consistent with the ability of a single antibody to useful in sandwich assays (1). Further work on this problem will be needed to further delineate the structures of the epitopes and whether more specific assays for CA125 can be devised. The molecular cloning of CA125 also opens up approaches to determining the function of CA125 and an understanding of its role in ovarian malignancy.

### REFERENCES

1. Bast, R. C., Jr., Klug, T. L., St John, E., Jenison, E., Niloff, J. M., Lazarus, H., Berkowitz, R. S., Leavitt, T., Griffiths, C. T., and Parker, L., et al. (1983) N. Engl. J. Med. 309, 883-887

2. Bast, R. C., Jr., Xu, F. -J., Yu, Y. H., Barnhill, S., Zhang, Z., and Mills, G. B. (1998) Int. J. Biol. Markers 13, 179-187

3. Menon, U. and Jacobs, I. J. (2000) Curr. Opin. Obstet. Gynecol. 12, 39-42

4. Lloyd, K. O., Yin, B. W. T., and Kudryashov, V. (1997) Int. J. Cancer 71, 842-850

5. Lloyd, K. O. and Yin, B. W. T. (2001) Tumor Biol. 22, 77-82

6. O'Brien, T. J. (1998) Int. J. Biol. Markers 13, 188-195

7. Yin, B. W. T. (2001) J. Biol. Chem. 276, 27371-27375

8. Yin, B. W. T. Dnistrian A., and Lloyd, K. O. (2002) Int. J. Cancer 98, 737-740

9. O'Brien T. J. Beard, J. B., Underwood, L. J., Dennis, R. A., Santin, A. D., and York, l. (2001) Tumor Biol. 22, 348-366

10. Kozak M. (1991) J. Biol. Chem. 266, 19867-19870 Gendler, S. J. and Spicert, A. P. (1995) Annu. Rev. Physiol. 57, 607-634

11. Perez-Villar, J. and Hill, R. L. (1999) J. Biol. Chem. 274, 31751-31754

12. Dessayn, J. -C., Buisine, M. -P., Porchet, N., Aubert, J. -P., and Laine, A. J. (1998) J. Biol. Chem. 273, 30157-30164

13. Nustad, K., Yenedin, Y. Lloyd, K. O., Shigemasa, K., de Bruijn, H. W. A. Jansson, B., Nilsson, O., O'Brien t. J. (2002) Tumor Biol.—in press

What is claimed is:

1. An isolated nucleic acid molecule comprising sequences encoding the CA125 protein or a portion thereof.

2. The gene encoding the CA125 protein.

3. The isolated nucleic acid molecule of claim 1 comprising sequence set forth in FIG. 6 and the corresponding CA125 protein comprising sequence set forth in FIG. 5.

4. The isolated nucleic acid molecule of claim 1 comprising sequence set forth in FIG. 7 and the corresponding CA125 protein sequence set forth in FIG. 8.

5. The nucleic acid of claim 1 comprising sequence set forth in FIG. 11.

6. The nucleic acid of claim 1 encoding protein comprising at least a portion of the amino acid sequence set forth in FIG. 12.

7. The gene of claim 2 comprising sequence set forth in FIG. 10.

8. The isolated nucleic acid molecules of claim 1, 2, 3, 4, 5, 6, or 7, wherein the nucleic acid is RNA, cDNA, genomic DNA, or synthetic DNA.

9. A vector comprising the nucleic acid molecule of claim 1, 2, 3, 4, 5, 6, 7, or 8.

10. The vector of claim 9, designated as pBK-CMV-B4 comprising sequence set forth in FIG. 6 and the corresponding CA125 protein comprising sequence set forth in FIG. 5.

11. The vector of claim 9, designated as pBKCMV-B30 comprising sequence set forth in FIG. 7 and the corresponding CA125 protein comprising sequence set forth in FIG. 8.

12. The vector of claim 9, designated as pCMV-Tag-B4 comprising sequence set forth in FIG. 6 and the corresponding CA125 protein comprising sequence set forth in FIG. 5.

13. The vector of claim 9, designated as pCMV-Tag-B30 comprising sequence set forth in FIG. 7 and the corresponding CA125 protein comprising sequence set forth in FIG. 8.

14. An expression system comprising the vector of claim 9.

15. The expression system of claim 14, wherein the system is a eukaryotic or prokaryotic system.

16. A method for producing CA125 protein comprising the expression system of claim 14.

17. An isolated nucleic acid molecule comprising sequence capable of specifically hybridizing to the sequences of claim 1 or 2.

18. The nucleic acid molecule of claim 17 capable of inhibiting the expression of the CA125 protein.

19. A method of inhibiting expression of CA125 inside a cell by vector-directed expression of an RNA able to hybridize with the RNA of CA125.

20. The nucleic acid molecule of claim 17 or 18 which is at least a 10 mer.

21. The nucleic acid molecule of claim 17 or 18 which is at least a 20 mer.

22. A method to detect ovarian cancer in a subject comprising steps of:
a) contacting the isolated nucleic acid molecule of claim 17 with RNA from a sample from the subject under conditions permitting the formation of a hybrid complex, and
b) detecting the hybrid complex, wherein a positive detection indicates the expression of the antigen and presence of cancer.

23. A method of monitoring ovarian cancer therapy in a subject comprising steps of:
a) contacting the isolated nucleic acid molecule of claim 17 with RNA from a sample from the subject under conditions permitting the formation of a hybrid complex, and
b) measuring the amount of the hybrid complex, wherein a decrease in the hybrid complex indicates the success of therapy.

24. A method for inhibiting the expression of the CA125 protein comprising contacting an appropriate amount of the nucleic acid molecule of claim 17 or 18 so that hybridization of the gene or transcript encoding the CA125 protein will occur, thereby inhibiting the expression of the protein.

25. A composition comprising the isolated nucleic acid molecule of claim 17 or 18.

26. A vaccine for a cancer which expresses CA125 protein comprising an appropriate amount of the isolated nucleic acid molecules of claim 1 or 2.

27. A vaccine for a cancer which expresses CA125 protein comprising an appropriate amount of an expression vector with the nucleic acid molecules which, when expressed, are capable of producing a product which induces an immune response to CA125 protein.

28. The vaccine of claim 27, wherein the nucleic acid molecule comprises sequences encoding human CA125 protein or a portion thereof.

29. The vaccine of claim 28, wherein the expressed human sequence is linked to a carrier.

30. The vaccine of claim 27, wherein the nucleic acid molecule comprises a nonhuman sequence.

31. The vaccine of claim 27, wherein the nucleic acid molecule comprises a primate sequence.

32. The vaccine of claim 27, wherein the nucleic acid molecule comprises a murine sequence.

33. The vaccine of claim 27, wherein the nucleic acid molecule comprises a synthetic sequence, which, when expressed, is capable of producing a product which induces an immune response to CA125 protein.

34. The vaccine of claim 33, wherein the sequence hybridizes with or is homologous to the sequences encoding human CA125 protein.

35. The vaccine of claims 26-34, further comprising a suitable adjuvant.

36. The vaccine of claims 26-34, wherein the adjuvant is an alum.

37. The vaccine of claims 26-36, wherein the cancer is an ovarian, pancreatic, breast, endometrial, or lung carcinoma.

38. A method to treat a cancer which expresses CA125 in a subject comprising administering to the subject an appropriate amount of the vaccine of claims 26-36.

39. The method of claim 38, wherein the cancer is an ovarian, pancreatic, breast, endometrial, or lung carcinoma.

40. A vaccine for a cancer which expresses CA125 comprising an appropriate amount of the expressed CA125 protein corresponding to the sequence in claim 1.

41. A vaccine for a cancer which expresses CA125 protein comprising an appropriate amount of a substance which induces an immune response to CA125 protein.

42. The vaccine of claim 41, wherein the substance is a polypeptide or a peptide.

43. The vaccine of claim 42, wherein the polypeptide comprises sequences encoding human CA125 protein or a portion thereof.

44. The vaccine of claim 43, wherein the expressed human sequence is linked to a carrier.

45. The vaccine of claim 41, wherein the polypeptide comprises a nonhuman sequence.

46. The vaccine of claim 45, wherein the polypeptide comprises a primate sequence.

47. The vaccine of claim 45, wherein the polypeptide comprises a murine sequence.

48. The vaccine of claim 42, wherein the polypeptide comprises a synthetic sequence, which, when expressed, is capable of producing a product which induces immune response to CA125 protein.

49. The vaccine of claims 40-48, further comprising a suitable adjuvant.

50. The vaccine of claim 49, wherein the adjuvant is an alum.

51. The vaccine of claims 40-50, wherein the expressed protein is conjugated to a protein carrier to increase the immunogenicity.

52. The vaccine of claims 40-51, wherein the cancer is an ovarian, pancreatic, breast, endometrial, or lung carcinoma.

53. A method to treat a cancer which expresses CA125 in a subject comprising administering to the subject an appropriate amount of the vaccine of claims 40-51.

54. A method to prevent a cancer which expresses CA125 in a subject comprising administering to the subject an appropriate amount of the vaccine of claims 40-51.

55. The method of claims 53 or 54, wherein the cancer is an ovarian, pancreatic, breast, endometrial, or lung carcinoma.

56. A method for the diagnosis of a cancer which expresses CA125 by detecting CA125-expressing cells in the blood or other fluids of patients based on the nucleic acid sequence which encodes CA125.

57. A method for monitoring the therapy of a cancer which expresses CA125 by measuring the expression of CA125-expressing cells in the blood or other fluids of patients based on the nucleic acid sequence which encodes CA125, a decrease of either the number of CA125-expressing cells or level of protein expression in the cell, indicating the success of the therapy.

58. The method of claim 56 or 57, wherein the detection is based on polymerase chain reaction with appropriate primers.

59. A method of producing CA125 protein comprising steps of:
a) constructing a vector adapted for expression in a cell which comprises the regulatory elements necessary for expression of nucleic acid in the cell operatively linked to the nucleic acid encoding the CA125 protein so as to permit expression thereof;
b) placing the cells of step (a) under conditions allowing the expression of the CA125 protein; and
c) recovering the CA125 protein so expressed.

60. The method of claim 59, wherein the cell type is selected from the group consisting of bacterial cells, yeast cells, insect cells, and mammalian cells.

61. The CA125 protein expressed by the method in claim 59 or 60.

62. A method for production of antibodies against CA125 protein using the protein of claim 61.

63. Antibodies produced by the method of claim 62.

64. A method for monitoring the therapy of cancer which expresses CA125 using the antibodies of claim 63.

65. A method of diagnosis of cancer which expresses CA125 using the antibodies of claim 63.

66. A method for determining the immunoreactive part of CA125 comprising contacting antibodies which are known to be reactive to CA125 with the protein of claim 61.

67. A transgenic nonhuman organism comprising the isolated nucleic acid molecule of claim 1 or 2.

68. A transgenic nonhuman mammal of claim 67.

69. A nonhuman organism, wherein the expression of CA125 is inhibited.

70. The nonhuman mammal of claim 69.

71. The nonhuman mammal of claim 70, wherein the mammal is a mouse.

72. A method for screening a compound for treatment of cancer which expresses CA125 protein comprising administering the compound to the transgenic nonhuman organism of claims 67-71, a decrease in expression of CA125 protein indicating that the compound may be useful for treatment of the cancer.
